SEQUENCE LISTING (69 sequences)

SEQ ID NO: 1 - protein, 303 aa, Homo sapiens
  PEPTIDE (1)...(303): human cdk4
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 2 - DNA, 1342 bp, Homo sapiens
  mRNA (1)...(1342): human cdk4 cDNA.
gtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtc

SEQ ID NO: 3 - protein, 303 aa, Homo sapiens; identical to SEQ ID NO: 1
  PEPTIDE (1)...(303): human cdk4

SEQ ID NO: 4 - DNA, 1399 bp, Homo sapiens
  mRNA (1)...(1399): human cdk4 cDNA
ctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataa

SEQ ID NO: 5 - protein, 303 aa, Homo sapiens; identical to SEQ ID NO: 1
  PEPTIDE (1)...(303): human cdk4

SEQ ID NO: 6 - DNA, 1441 bp, Homo sapiens
  mRNA (1)...(1441): human cdk4 cDNA
gccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtctttttttttttttt

SEQ ID NO: 7 - protein, 303 aa, Mus musculus
  PEPTIDE (1)...(303): Balb/C mouse cdk4
MAATRYEPVAEIGVGAYGTVYKARDPHSGHFVALKSVRVPNGGAAGGGLPVSTVREVALLRRLEAFEHPNVVRLMDVCATSRTDRDIKVTLVFEHIDQDLRTYLDKAPPPGLPVETIKDLMRQFLSGLDFLHANCIVHRDLKPENILVTSNGTVKLADFGLARIYSYQMALTPVVVTLWYRAPEVLLQSTYATPVDMWSVGCIFAEMFRRKPLFCGNSEADQLGKIFDLIGLPPEDDWPREVSLPRGAFAPRGPRPVQSVVPEMEESGAQLLLEMLTFNPHKRISAFRALQHSYLHKEESDAE

SEQ ID NO: 8 - DNA, 912 bp, Mus musculus
  mRNA (1)...(912): Balb/C mouse cdk4 cDNA
atggctgccactcgatatgaacccgtggctgaaattggtgtcggtgcctatgggacggtgtacaaagcccgagatccccacagtggccactttgtggccctcaagagtgtgagagttcctaatggaggagcagctggagggggccttcccgtcagcacagttcgtgaggtggccttgttaaggaggctggaggcctttgaacatcccaatgttgtacggctgatggatgtctgtgctacttcccgaactgatcgggacatcaaggtcaccctagtgtttgagcatatagaccaggacctgaggacatacctggacaaagcacctccaccgggcctgccggttgagaccattaaggatctaatgcgtcagtttctaagcggcctggattttcttcatgcaaactgcattgttcaccgggacctgaagccagagaacattctagtgacaagtaatgggaccgtcaagctggctgactttggcctagctagaatctacagctaccagatggccctcacgcctgtggtggttacgctctggtaccgagctcctgaagttcttctgcagtctacatacgcaacacccgtggacatgtggagcgttggctgtatctttgcagagatgttccgtcggaagcctctcttctgtggaaactctgaagccgaccagttggggaaaatctttgatctcattggattgcctccagaagacgactggcctcgagaggtatctctacctcgaggagcctttgcccccagagggcctcggccagtgcagtcagtggtgccagagatggaggagtctggagcgcagctgctactggaaatgctgacctttaacccacataagcgaatctctgccttccgagccctgcagcactcctacctgcacaaggaggaaagcgacgcagagtga

SEQ ID NO: 9 - protein, 303 aa, Rattus norvegicus
  PEPTIDE (1)...(303): rat cdk4
MATTRYEPVAEIGVGAYGTVYKARDPHSGHFVALKSVRVPNGGAAGGGLPVSTVREVALLRRLEAFEHPNVVRLMDVCATSRTDRDIKVTLVFEHIDQDLRTYLDKAPPPGLPVETIKDLMRQFLSGLDFLHANCIVHRDLKPENILVTSNGTVKLADFGLARIYSYQMALTPVVVTLWYRAPEVLLQSTYATPVDMWSVGCIFAEMFRRKPLFCGNSEADQLGKIFDLIGLPPEDDWPREVSLPRGAFSPRGPRPVQSVVPEMEESGAQLLLEMLTFNPLKRISAFRALQHSYLHKEESDPE

SEQ ID NO: 10 - DNA, 1232 bp, Rattus norvegicus
  mRNA (1)...(1232): rat cdk4 cDNA
ccgaagcgtccagctgcccgggaccgatccccggtgtatggcgccgcagaaacggctcccgggcccagataaagggcacctccgcagctcttggccgagagatcccctgctccgagaatggctaccactcgatatgaacccgtggctgaaattggtgtcggtgcctatgggacggtgtacaaagcccgagatccccacagtggccactttgtggctctcaagagtgtgagagttcctaatggaggagcagctggagggggccttcccgtcagcacagttcgtgaggtggccttgttaagaaggctggaggcctttgaacatcccaatgttgtacggctgatggatgtctgtgctacttcccgaactgatcgggacatcaaggtcaccttagtgtttgagcatatagaccaggacctacggacatacctggacaaagcacctccgccgggcttgcctgttgagaccattaaggatctgatgcgccagtttctaagcggcctagatttccttcatgcaaactgcattgttcaccgggacctgaagccagagaacattctagtgacaagtaatgggacagttaagctggccgactttggcctagccagaatctacagctaccagatggccctcacgcctgtggttgttacgctctggtaccgggctcctgaagttcttctgcagtctacatatgcaacgcctgtggatatgtggagtgttggctgtatcttcgcagagatgtttcgccggaagcctctcttctgtgggaactctgaggctgaccagctgggcaaaatctttgatctcattggattgcctccagaagacgactggcctcgagaggtctctcttcctcgaggagccttttcccccagaggacctcggccagtgcagtcagtggtgccggagatggaggaatctggagcgcagttgctgctggaaatgctgacctttaatccacttaagcgaatctctgccttccgagccctgcagcactcttacctgcacaaggaggaaagtgacccggagtgaggagaggggctgccttttccagtttccggacgctgtgtggaaagaaccttgctgaagtggcagcctctgccttcccctgaggctatggagagtcctccggtttagccttaaatgacaattccctacctctccttatgaggtttaccccacccccaccctcctgtttctctacactaaagggcaggcattatctgtcttcttcc

SEQ ID NO: 11 - protein, 303 aa, Sus scrofa
MATSRYEPVAEIGVGAYGTVYKARDPHSGHFVALKSVRVPNGGGAGGGLPISTVREVALLRRLEAFEHPNVVRLMDVCATARTDRETKVTLVFEHVDQDLRTYLDKAPPPGLPVETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFSPRGPRPVQSVVPEMEESGAQLLLEMLTFNPHKRISAFRALQHSYLHKAEGNPE

SEQ ID NO: 12 - DNA, 912 bp, Sus scrofa
  CDS (1)...(912): domestic pig cdk4 coding sequence
atggctacctcccggtatgaaccagtggcggagattggtgttggtgcctatgggaccgtgtacaaagcacgggatccccacagtggccactttgtggccctcaagagcgtaagagtccccaatggaggaggtgctggagggggcctgcccatcagcacggttcgtgaagtggccttactgcgccggctggaggctttcgagcatcccaatgttgtccggctgatggatgtctgtgccactgcccgaactgatcgagagaccaaagtgaccctggtgtttgagcatgtggaccaagatctaaggacatatctggacaaggcacccccaccaggcttgccggtggagaccatcaaggatctgatgcgtcagtttctaagaggcctagatttccttcatgccaactgcatcgttcatcgagacctgaagccagagaacattctggttacaagtggtgggacagtcaagctggctgactttggcctggccagaatctacagctaccagatggcacttacacctgtggtggttacactctggtaccgtgctccagaagttcttttgcagtctacgtatgcaacacctgtggacatgtggagcgttggctgtatcttcgcagagatgtttcgtcgaaagcctctcttctgtggaaactctgaagctgaccagttaggcaaaatctttgacctgattgggctgcccccagaggatgactggccccgagatgtgtctctaccccgaggagccttttcccccagagggccccgcccagtgcagtcggtggtacctgagatggaggagtctggagcacagctgctactggagatgctgacttttaacccacacaagcgaatctctgccttccgagccctgcagcactcttatctacataaggcagaaggtaacccagagtga

SEQ ID NO: 13 - protein, 319 aa, Xenopus laevis
MSKEMKGQYEPVAEIGVGAYGTVYKARDLQSGKFVALKNVRVQTNENGLPLSTVREVTLLKRLEHFDHPNIVKLMDVCASARTDRETKVTLVFEHVDQDLKTYLSKVPPPGLPLETIKDLMKQFLSGLEFLHLNCIVHRDLKPENILVTSGGQVKLADFGLARIYSCQMALTPVVVTLWYRAPEVLLQSTYATPVDVWSAGCIFAEMFKRKPLFCGNSEADQLCKIFDIIGLPSEEEWPVDVTLPRSAFSPRTQQPVDKFVPEIDAMGADLLLAMLTFSPQKRISASDALLHPFFADDPQACSKQEHFTHICTATDEVK

SEQ ID NO: 14 - DNA, 2258 bp, Xenopus laevis
  mRNA (1)...(2258): X. laevis cDNA
ggcacgagcggcacgagcatcctcctcatgctagcgtgcagcagtgcaaggcatggcgtgaatgcgggcgcttccatgtcaggagatcagcgctgtgaggttcccgtgctattcaggacagggggttcttgagaagaataccagtgtggaatatgtcaaaagaaatgaagggtcaatatgaacctgtcgcagagattggcgtcggggcttacggcacggtatacaaggccagggaccttcaaagtggaaagtttgttgccctcaaaaatgtgcgggtgcaaaccaacgagaatggactccccctctctacggtcagggaagtgactttattgaaacgcctggagcattttgaccatccaaacattgtaaaacttatggatgtttgtgcttctgcccgaactgaccgagagactaaagtgacgctggtgtttgaacatgtggaccaagacctgaagacttacttgagcaaagtcccacctccaggtcttccactggagactattaaggacctcatgaagcagttcctttcgggcctggaattcctccatttgaattgcattgtgcaccgagaccttaaaccggagaatatcttggtgacaagtgggggacaggtgaaacttgccgactttggcctagcgaggatatacagctgtcagatggctctcacgcccgtggtggtgacgttgtggtacagggccccagaagtcctgcttcagtccacgtatgccacgccggtagatgtgtggagcgcgggctgcatcttcgcagagatgttcaagcgaaaacccctgttctgcggcaattcagaagctgaccagttgtgcaagatatttgatattattgggcttccttctgaagaggagtggccggtggatgttactttgccacgttctgccttttccccgaggactcagcagccagtggacaagtttgtgccggaaatcgatgccatgggagccgatcttcttctggccatgctgaccttcagccctcaaaaacgtatttccgcctccgatgctctgctccacccctttttcgctgacgatccccaggcctgctccaaacaagaacactttactcatatctgcaccgcaacggatgaagtgaaatgaaacggcatccctgcctgggggctccagggtcggcgcagatgtgggcacattatttgctttttctctgccttaaagggagttctcagcgggaggggaaagcaaataagcgataaagtgaccatgtgatccttgtaaaacatttccacttcctggttgtaatggattttatgctgcaaacgtcactgtgaccgacgcaagattccgctgttcagatcagcgccgtttcgcatttgtgccttatcggtccttacatttatggtttggggagctcccttaaccagagacgcacaataagctacactatcctcattatttctgttctcaccgcattgatggtgtcaaatctcggctttaccacaatttttatcacggtgctctttctggggtaaaaaaacaaaaaagacttattgatcatgtgctaaaatttagcgcacgccagactgttcgagagcccgacaaataatttctaccatttttgtactgttttacaataatgttttatattggattttcaagggtatttctattgcgctggggtggggtaaaaattgacctgcggctattttgttcctatggtaaaaacatggcagtcttaacccactagggatccacggaatccgctattttttttgttaggctgaatcctctacaaaagattctgcaaaatctgaacccaaatttgcaagttcatattggataaaaaccaagcatttcacattcagttattgagagcttaaaagacaaatggccatgagggttaggattcggccgaatgttaatgtttcattaaagtgctaggttttaaccaaagcaacaaccaaaacttggccttgttgcatccctacaactgacatcttgatctttcatgctggtgaatgttggacggtcatgggcccattgtgggtataatttgttcaatttggagcctgcctcgctaaatcctttaatgcagaattctttccttttctgaactgcccttgtttgtctgttgacattgaaaccccattatcgtcgttggttcaactgcgcacgctgtcgttctgaatgttacagttcagctttaaagtctactcataaagtgaagtttaaagcgggtgggaattgccagatttagtgtgagggtgaaaaaaaaaaaaaactcgtgcc

SEQ ID NO: 15 - protein, 303 aa, Homo sapiens [sequence omitted]

SEQ ID NO: 16 - DNA, 1351 bp, Homo sapiens
  mRNA (1)...(1351): Human cdk4R24C mutant
gtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcctgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaaaaaagtcgacgcggccgcgaattc

SEQ ID NO: 17 - protein, 326 aa, Homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 18 - DNA, 1247 bp, Homo sapiens
gtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaa

SEQ ID NO: 19 - protein, 326 aa, Homo sapiens
MEKDGLCRADQQYECVAEIGEGAYGKVFKACDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 20 - DNA, 1247 bp, Homo sapiens
gtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcctgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaa

SEQ ID NO: 21 - protein, 326 aa, Mus musculus (Balb/c)
MEKDSLSRADQQYECVAEIGEGAYGKVFKARDLKNGGRFVALKRVRVQTSEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDIIGLPGEEDWPRDVALPRQAFHSKSAQPIEKFVTDIDELGKDLLLKCLTFNPAKRISAYGALNHPYFQDLERYKDNLNSHLPSNQSTSELNTA

SEQ ID NO: 22 - DNA, 2470 bp, Mus musculus (Balb/C)
ggcagcgagtgagcaccccggttccactgtgccgcacccgcagcctgaagccagcatggagaaggacagcctgagtcgcgccgatcagcagtatgagtgcgtggcggagatcggcgaaggcgcctatgggaaggtgttcaaggcccgcgacctgaagaacggcggccgcttcgtggctctgaagcgcgtgcgagtgcagaccagtgaggagggcatgccgctctccaccatccgcgaggtggcggtgctgaggcacctggagaccttcgagcaccccaacgtggtcaggttgtttgatgtgtgcacagtgtcacggacggacagagaaaccaagcttacactagtgtttgagcatgttgatcaagacttgaccacttacttggataaagttccagagcccggcgtacccacagaaaccataaaggatatgatgtttcagcttctccgaggtctggactttcttcattctcacagagtagtgcatcgtgatctgaaaccgcagaacattctggtgaccagcagtggacagataaagctggctgactttggccttgcccgcatctatagttttcagatggcccttacctcggtggtcgtcacgctgtggtaccgagccccagaagtcctgctccagtccagctatgccacccctgtggacctctggagtgtcggttgcatctttgcagaaatgtttcgcagaaagcctctttttcgtggaagttcagacgtggatcaactaggaaaaatcttggacatcattggactcccaggagaggaagactggcctagggacgtggcccttccccggcaggcttttcattccaaatctgctcaacccatcgagaagtttgtgacagatattgacgaactaggcaaagacctacttctgaaatgcctgacgtttaatccagctaaaaggatatccgcctacggcgccctgaatcacccgtacttccaagatctggagagatacaaggacaacctgaactctcacctgccatccaaccagagcacctcggagctgaacacagcctgaggttccacggggatgcccatgagctcgtcatctgaacacattggcggctgcgagtcccctaagcaagcctctcagagcagttgaagattgctggctgccaaccttctggctgccagcttctgggtgggctctgccttaccaaggaaaccacctagtttactgttcagagatcaatgcaagggtgattgcagctttatgttcgtttgtacacttgtttgttttgtctgtttgtttcaagaacctggaaaacttccagaagaagagaagctgctgaccaattgtgctgccatttcgttttctaaccttgaatgctgccagtgtagggtgggaatccaggcccagctgagttatgatgtaatccgcctgcagctgctgggcctgctttggtacttgtgagtgtgtgtgcatgcgtatgtgtgtgtaagagagaagaggaggggagagaaagacccctgatctcgtcaagtgttactttttttttgtagaaaacaagaataattgagttttaaagagtagaggtgactgatagtaagaagggcttgctcagtgaaaggtgattcacaatggagtcttgttaggaaggttggacctaagtcctcagagttgccttcctgtccaaaagcttttgctagcagtaaacaataaaggtttagatgccacaaaaaatggggggaaccgcaatattttttaagagactttttaaggcatacatcttctatttactctttggaaagctgaacttaatgtgtcccaggccctatatatagtacagtatgtacttaattgtttctttggggaaagatgctataagtatcttattacttgcaatacatttaaggagtgagtgtacctcagataggttttaaagatagagagcacctgttttctggtgtgagatgttatcattttcttcacgtctcttgataccttgataccttgtcaccttagggaatcacttcctgctctgactagaggcgggaataccatctagctgtctccaccacccaccatggcgcatctgccttgtgctgccttgtgtagtgcgaagctctcaaccaccagcacttctaattcattttcctgccactgcctggctaacgacagatggcccagctgccccaatcccacacccgcttgcacgcttaccgtctttcaccgaatgctttgggcgtaggctcccattccgaaaccctaacagtatccccttgtgcctttgtaatacagtcttccccctgccgcagctgaggtcacctaggcagtgaagagtgcttgttctgtgtgtgtatagactactaccgactgtcacttggtgtttcctatctttaagtgtatgttgtcagtgtaatgtctgaggaaatgtcttttcctctcttctagagataactacttactctctaaagtgatctctctgtctgtccgcaggatgtgtttctg

SEQ ID NO: 23 - protein, 111 aa, Rattus Norvegicus
GKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDF

SEQ ID NO: 24 - DNA, 333 bp, Rattus Norvegicus
gggaaggtgttcaaggcccgcgacctgaagaacggcggccgcttcgtggctctgaagcgcgtgcgagtgcagaccggagaggagggcatgccgctctccaccatccgcgaggtggcggtgctgaggcacctggagacctttgagcaccccaacgtggtcaggttgtttgacgtgtgcacagtgtcacggacagacagagaaactaaacttacgctagtgtttgagcatgttgatcaagacttgaccacttacttggataaagttccagaacccggtgtgcccacagagaccataaaggatatgatgtttcagcttctccgaggtctggacttt

SEQ ID NO: 25 - protein, 326 aa, Gallus gallus
MDKDGTNLADQQYECVAEIGEGAYGKVFKARDLKNGGRFVALKRVRVQTSEEGMPLSTIREVAVLRHLETFEHPNVVRLFDVCTVSRTDRETKLTLVFEHVDQDLTTYLDKVPEPGVPTETIKDMMLQLFRGLDFLHSHRVVHRDLKPQNILVTSSGQIKLADFGLARIYSFQMALTSVVVTLWYRAPEVLLQSSYATPVDLWSVGCIFAEMFRRKPLFRGNSDVDQLGKIFDVIGLPEEEDWPNDVALPRNAFASRPAQPIEKFVPDIDDMGKDLLLKCLAFNPAKRISAYAALSHPYFHDLEKCKENLDSHMSSSQNSSEVNAS

SEQ ID NO: 26 - DNA, 1453 bp, Gallus gallus
gccgccggcggagcgggcggccgcggcgcgcaccagatctagctgccttcgcacagaccatgctcccaaaggctgtatgatcatttctggtgactgccagcggaggctcactgggacccgtgcagcagagaaggcggacgaggggttcctgctgcccctcgggcagaggtaccggcgtgtggccgacccacagcgaggtgccagcttcagaagccggcagagacgagagctgccgtgaggtgctccggacccgggaggaagcggagcggcggcagcatggacaaggacggcaccaacctggccgaccaacagtatgagtgcgtggctgagatcggcgaaggagcctacgggaaggtgttcaaggcccgcgacctgaagaatggcggccgcttcgtggcgctgaagcgggtgcgggtgcagaccagcgaggagggcatgccgctgtccaccatccgggaggtggccgtcctgaggcacctggagaccttcgagcaccccaacgtggtcagattgtttgatgtgtgcaccgtgtcacgaacagacagagaaaccaagttaacgttggtgtttgaacatgtggatcaagacttgactacttacttggataaagttccagagcctggagtgcctactgaaactataaaggatatgatgcttcagctgtttcggggactggattttctgcattcacatcgtgtggtgcatcgggacctgaaaccccagaatatccttgtaaccagcagtgggcagataaagttagctgactttggacttgcacgaatctacagttttcagatggctcttacatcagtggttgttactttgtggtatagagctcctgaagttttgcttcagtccagctatgcaacaccagttgatctttggagtgttggttgcatatttgcagaaatgttccgtcgaaaaccactcttccgtggaaattcagatgttgatcagctaggaaaaatctttgatgtaattggactcccagaagaagaggactggcctaatgatgtggcccttccaagaaatgcttttgcttccagacccgcacaacctattgaaaaatttgtaccagatattgatgacatgggcaaagacttgcttcttaaatgcttagcgttcaatccagccaagagaatatctgcctatgctgccctgtctcacccctatttccatgatctggagaaatgcaaggagaatctggactctcacatgtcatccagccaaaactccagtgaggtgaacgcatcataatgctgactgaagacgaacctatgaatgaacaagatgtgtagctgacaaggccactgtcccatacaaaccacatggctgttctagtgaagatcattgtttggaggttttacacacctgtctttcattttgggattgcgatgtgcttatccaaggaaatcaacgtagtttactttcatcagagcaatgcaagggccag

SEQ ID NO: 27 - protein, 298 aa, Homo sapiens
  VARIANT (1)...(298): Xaa = Any Amino Acid
MENFQKVEKIGEGTYGVVYKARNKLTGEVVALKKIRXDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 28 - DNA, 1297 bp, Homo sapiens
  misc_feature (1)...(1297): n = A,T,C or G
cgttggccaaattgacaagagcgagaggtatactgcgttccatcccgaccnggggccacggtactgggccctgtttccccctcctcggcccccgagagccagggtccgccttctgcagggttcccaggcccccgctccagggccgggctgacccgactcgctggcgcttcatggagaacttccaaaaggtggaaaagatcggagagggcacgtacggagttgtgtacaaagccagaaacaagttgacgggagaggtggtggcgcttaagaaaatccgnntggacactgagactgagggtgtgcccagtactgccatccgagagatctctctgcttaaggagcttaaccatcctaatattgtcaagctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtggtgaccctgtggtaccgagctcctgaaatcctcctgggctgcaaatattattccacagctgtggacatctggagcctgggctgcatctttgctgagatggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactctgatagccttcttgaagcccccagccctaatctcaccctctcctccagtgtgggcttgaccaggcttggccttgggctatttggactcaggtgggccctctgaacttgccttaaacactcaccttctagtcttggccagccaactctgggaatacaggggtgaaaggggggaaccagtgaaaatgaaaggaagtttcagtattagattgcacttaagttagcctccaccaccc

SEQ ID NO: 29 - protein, 298 aa, Homo sapiens
  VARIANT (1)...(298): Xaa = Any Amino Acid
MENFQKVEKIGEGTYGVVYKACNKLTGEVVALKKIRXDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 30 - DNA, 1297 bp, Artificial Sequence
  Mutation
cgttggccaaattgacaagagcgagaggtatactgcgttccatcccgaccnggggccacggtactgggccctgtttccccctcctcggcccccgagagccagggtccgccttctgcagggttcccaggcccccgctccagggccgggctgacccgactcgctggcgcttcatggagaacttccaaaaggtggaaaagatcggagagggcacgtacggagttgtgtacaaagcctgtaacaagttgacgggagaggtggtggcgcttaagaaaatccgnntggacactgagactgagggtgtgcccagtactgccatccgagagatctctctgcttaaggagcttaaccatcctaatattgtcaagctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtggtgaccctgtggtaccgagctcctgaaatcctcctgggctgcaaatattattccacagctgtggacatctggagcctgggctgcatctttgctgagatggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactctgatagccttcttgaagcccccagccctaatctcaccctctcctccagtgtgggcttgaccaggcttggccttgggctatttggactcaggtgggccctctgaacttgccttaaacactcaccttctagtcttggccagccaactctgggaatacaggggtgaaaggggggaaccagtgaaaatgaaaggaagtttcagtattagattgcacttaagttagcctccaccaccc

SEQ ID NO: 31 - protein, 298 aa, Mus musculus
MENFQKVEKIGEGTYGVVYKAKNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 32 - DNA, 1708 bp, Mus musculus
caagggctgagctctccttgcgttccatcccgagctgggactgccgtgctcacccggggtcccccaggtcctccgctccgagtgtcgggcccgctttcgtcagggttcccgggccccgctcccgggggcctgagccgcctcactagcgctccatggagaactttcaaaaggtggagaagattggagagggcacgtacggagtggtgtacaaagccaaaaacaagttgacgggagaagttgtggcgcttaagaagatccggctcgacactgagactgaaggtgtacccagtactgccatccgagagatctctctccttaaggaacttaatcaccctaatatcgtcaagctgctggatgtcatccacacagaaaataagctttatctggtttttgaatttctgcaccaggacctcaagaaattcatggatgcctctgctctcacgggcattcctcttcccctcatcaagagctatctgttccagctgctccagggcctggctttctgccattctcaccgtgtccttcaccgagaccttaagccccagaacctgcttatcaatgcagaggggtccatcaagctggcagactttggactagcaagagcctttggagtccctgtccgaacttacactcatgaggtggtgaccctgtggtaccgagcacctgaaattcttctgggctgcaagtactactccacagccgtggatatctggagcctgggctgcatctttgctgaaatggtgacccgcagggccctattccctggagattctgagattgaccaactcttccggatctttcggactctggggaccccagatgaggtggtttggccaggagttacttctatgcctgattataagccaagtttccccaagtgggctcggcaagattttagcaaagttgtgcctcccctggatgaagatggacggagcttgttatcgcaaatgctgcactatgaccccaacaagcggatttcagccaaagcagccctggctcaccctttcttccaggatgtaactaaaccagtgccccaccttcggctctgatgcccttcccaaagcccttttcacccgtggtctgacttgaccctgggccttttggacacaggcaagaagagccaggagggcacagggcttgcacgtcactctggtctgttcatcgtggttcacagggcaaggtgaaagacacttgaatttctctttttagcaatcttactgttttctggtgctgactgctacacccaggactttgccctcactaagcaaatgtaccaccaatgagccaggttcctagccttccttttggaagctcagttcttgagttgtcagagggcctcattgccgtcctcctctgagagcagtgatgcaggccaggagacatcatttgagaatgctgatgcttttgtaaggctttgacctggttaggtcattgggggaaattttctataaaagagtgaacaattatatttatatttcaagttaaagtagtttggatactttagtggttttgttgttgtttcttctttttccttttttttttttttttgtcagtgtggatggatttgttgccatgtgcactttgggattttgtaattgttaaaagaaaatatttcttttatgattttcttctcccactccctgtcctccagtgttctgttaatattatttgtaatttagtttgtaaattcattaaaagaaaatattct

SEQ ID NO: 33 - protein, 346 aa, Mus musculus
MENFQKVEKIGEGTYGVVYKAKNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMHLVCTQHHAKCCGEHRRNGRHSLCPLCSYLEVAASQGGGMTAVSAPHPVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 34 - DNA, 1380 bp, Mus musculus
tcccgagctggggactgccgggctcacccggggtcccccaggtcctccgctccgagggtcgggcccgctttcgtcagggttcccgggccccgctcccgggggcctgagccgcctcactagcgctccatggagaacttccaaaaggtggagaagattggagagggcacgtacggagtggtgtacaaagccaaaaacaagttgacgggagaagttgtggcgcttaagaagatccggctcgacactgagactgaaggtgtacccagtactgccatccgagagatctctctccttaaggaacttaatcaccctaatatcgtcaagctgctggatgtcatccacacagaaaataagctttatctggtttttgaatttctgcaccaggacctcaagaaattcatggatgcctctgctctcacgggcattcctcttcccctcatcaagagctatctgttccagctgctccagggcctggctttctgccattctcaccgtgtccttcaccgagaccttaagccccagaacctgcttatcaatgcagaggggtccatcaagctggcagactttggactagcaagagcctttggagtccctgtccgaacttacactcatgaggtggtgaccctgtggtaccgagcacctgaaattcttctgggctgcaagtactactccacagccgtggatatctggagcctgggctgcatctttgctgaaatgcacctagtgtgtacccagcaccatgctaagtgctgtggggaacacagaagaaatggaagacacagtctctgcccgctgtgctcctatctagaagtggctgcatcacaaggaggggggatgaccgcagtgtctgccccacaccccgtgacccgcagggccctattccctggagattctgagattgaccaactcttccggatctttcggactctggggaccccagatgaggtggtttggccaggagttacttctatgcctgattataagccaagtttccccaagtgggctcggcaagattttagcaaagttgtgcctcccctggatgaagatggacggagcttgttatcgcaaatgctgcactatgaccccaacaagcggatttcagccaaagcagccctggctcaccctttcttccaggatgtaactaaaccagtgccccaccttcggctctgatgcccttcccaaagcccttttcacccgtggtctgacttgaccctgggccttttggacacaggtcagccttctgatgttttctggctgtcttagcatccgccttttctcttgccagccagttctggggattcagaggtgcagggaagggttagggaaaagggggcaaggttttgggaattagatgcacttaacccggttccaccagttctccct

SEQ ID NO: 35 - protein, 298 aa, Mesocricetus auratus
MENFQKVEKIGEGTYGVVYKAKNKLTGEVVALKKIRLDTETEGVPSTAIREISLLKELNHPNIVKLLDVIHTENKLYLVFELLHQDLKKFMDASAVTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 36 - DNA, 1065 bp, Mesocricetus auratus
catcccgagctggggctgccgtgctcaccttggcccccaggtcctccgcccctgagtgtcgggcccgctttctttagggttcccgggccccgctcccgggggcctgagccgccgcattagcgctccatggagaacttccaaaaggtggagaagatcggagagggcacgtacggagtggtgtacaaagccaaaaacaagttgacgggagaagtggtggcgcttaagaaaatccggctcgacactgagactgaaggtgtgcccagtactgccatccgagagatctctctgcttaaggaacttaatcaccctaatattgtcaagctgctggatgtcatccacacagaaaataagctttacctggttttcgagcttctgcaccaggacctcaagaaatttatggatgcctctgctgtcactggcatccctcttcccctcatcaagagctatctgttccagctgctccagggcctagcattctgccattctcaccgggtcctgcaccgagaccttaagccccagaatctgcttatcaatgcagagggatccatcaagttggcagactttggactagcaagagcctttggagtccccgttcgtacttatactcatgaggtggtgaccttgtggtaccgagcacctgaaatccttctaggctgcaagtactactccacagccgtggatatctggagcctgggctgcatcttcgctgaaatggtgacccggcgggccctgtttcctggagattctgagattgatcaactcttccggatctttcggactctggggaccccagatgaggtggtttggccaggagttacttctatgcctgattataagccgagtttccccaagtgggctcggcaagattttagcaaagttgtgcctccactggatgaagatggacggagcctgttatcgcaaatgctgcactatgaccccaacaagcggatttcagccaaagcagccctggcccaccctttcttccaggatgtgactaagccagtaccccaccttcggctgtgatgtccttcccaaagccctcctcacccttgggtcttgacttga

SEQ ID NO: 37 - protein, 346 aa, Mesocricetus auratus
MENFQKVEKIGEGTYGVVYKAKNKLTGEVVALKKIRLDTETEGVPSTAIREISLLKELNHPNIVKLLDVIHTENKLYLVFELLHQDLKKFMDASAVTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMHLVCTQHHAKCCGEHRRNGRHSLCPLCSYLEVAASQGGGMTAVSTPYPVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 38 - DNA, 1209 bp, Mesocricetus auratus
catcccgagctggggctgccgtgctcaccttggcccccaggtcctccgcccctgagtgtcgggcccgctttctttagggttcccgggccccgctcccgggggcctgagccgccgcattagcgctccatggagaacttccaaaaggtggagaagatcggagagggcacgtacggagtggtgtacaaagccaaaaacaagttgacgggagaagtggtggcgcttaagaaaatccggctcgacactgagactgaaggtgtgcccagtactgccatccgagagatctctctgcttaaggaacttaatcaccctaatattgtcaagctgctggatgtcatccacacagaaaataagctttacctggttttcgagcttctgcaccaggacctcaagaaatttatggatgcctctgctgtcactggcatccctcttcccctcatcaagagctatctgttccagctgctccagggcctagcattctgccattctcaccgggtcctgcaccgagaccttaagccccagaatctgcttatcaatgcagagggatccatcaagttggcagactttggactagcaagagcctttggagtccccgttcgtacttatactcatgaggtggtgaccttgtggtaccgagcacctgaaatccttctaggctgcaagtactactccacagccgtggatatctggagcctgggctgcatcttcgctgaaatgcacctagtgtgtacccagcaccatgctaagtgctgtggggaacacagaagaaatggaagacacagtctctgcccgctgtgctcctatctagaagtggctgcgtcacaaggaggggggatgaccgcagtgtctaccccataccccgtgacccggcgggccctgtttcctggagattctgagattgatcaactcttccggatctttcggactctggggaccccagatgaggtggtttggccaggagttacttctatgcctgattataagccgagtttccccaagtgggctcggcaagattttagcaaagttgtgcctccactggatgaagatggacggagcctgttatcgcaaatgctgcactatgaccccaacaagcggatttcagccaaagcagccctggcccaccctttcttccaggatgtgactaagccagtaccccaccttcggctgtgatgtccttcccaaagccctcctcacccttgggtcttgacttga

SEQ ID NO: 39 - protein, 298 aa, Cricetulus griseus
MENFQKVEKIGEGTYGVVYKAKNKLTGEVVALKKIRLDTETEGVPSTAIREISLLKELNHPNIVKLLDVIHTENKLYLVFEFLHQDLKKFMDASAVTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 40 - DNA, 2213 bp, Cricetulus griseus
attgtcaagggctgagctttgcttgcgttccatccagagctggggctgcggtgctcaccttggcccccaggtcctccgccccgagtgttgggcccgctttcttcagggttcccgggccccgctcccgggggcctgagccgccgcactagtgctccatggagaacttccaaaaggtggagaagatcggagagggcacgtacggagtggtgtacaaagccaaaaacaagttgacgggagaagtggtggcgcttaagaaaatccggctcgacactgaaactgaaggtgtgcccagtactgccatccgagagatctctctgcttaaggaacttaatcaccctaatattgtcaagctgctagatgtcatccacacggaaaataagctttacttggtttttgaatttctgcaccaggacctcaagaaatttatggatgcctctgctgtcactggcattcctcttcccctcatcaagagctatttgttccagctgctccagggcctagcattctgccattctcaccgggtcctgcaccgagaccttaagccccagaatctgcttatcaatgcagaggggtccatcaagttggcagactttggactagcaagagcctttggagtccctgttcgtacttatactcatgaggtggtgaccctgtggtaccgagcacctgaaatccttctaggctgcaagtactactccacagccgtggatatctggagtctgggctgcatctttgctgaaatggtgacccggcgggccctgtttcccggagattctgagatcgaccaactcttccggatcttccgaactctggggacccctgatgaggtggtttggccgggagtcacttctatgcctgattataagccgagtttcccgaagtgggctcggcaagattttagcaaagttgtgcctccactggatgaagatgggcggagcttgttatcgcaaatgctgcactatgaccccaacaagcggatttcagccaaagcagccctggcccaccctttcttccaggatgtgactaagccagtaccccaccttcggctgtgatgtccttcccaaagccttcctcacccttggtctgacttgactctgggccttttggacacaggtcagccttctactcttttctggctgtcttagcactcactttttctcttggccagccagttcccgggactcagaggtgcagggaaagatgaggtgacaggaaaaagttttggtatcagatgcacttaacccagcttccactaccttctctccctcttagtcatcactgagggttggtatgcaaaacaaaacaaaactgaccattctctacccacccccatgtcaagtttggccatcccagtcttataaattattgtgtcccatgtttaggaagacacaccccagacctcctggtgccactgttttataaaggccaactgataagttgggacatttaggaaccaagccacagaggtctattttaatgaattagatgaaaaaaaaaatagatccaaacagtttataccttagttgtagtgtctggcctcgcccgacaatcaaggaggcccagctggagaagcagaagaaatgttccctttcagcactctgtctaccttctgcagggcaagaagtcaggagggggatagggattggtctgttcaccataattcacaaggcaaggtgaaagacactggaatctttcttttagtaattttgttttctggtgctaacgactgaacacaggacctcgccctcactaagcaaatgcaccacccgtatgccagattttctagctcctctttttggcgttttagctcttcagtttgtcagaggtccctgttgccatcttcttcctctgagagtcacccctgactccatagtggaagtgaagctttagatgtcactttgaaaggctttgacctggtcaggtcatcgggggacattttctataaaagagttaacaattatatttatatttcaagttatagtagtttggatattttagtgttttgatttttgtttttttggggggcggggcagtgtggatggatttgttgccatgtgcactttggaattttgtaatgcaattgttgaaagaaaatatttcttttatgatcttctccctctaccccttgtcctccagtgttctgttaatatttatttgtaatttagtttgtaattcattaaaagaaattattctcaaaaaaaaaaaaaaaaaaa

SEQ ID NO: 41 - protein, 346 aa, Cricetulus griseus
MENFQKVEKIGEGTYGVVYKAKNKLTGEVVALKKIRLDTETEGVPSTAIREISLLKELNHPNIVKLLDVIHTENKLYLVFEFLHQDLKKFMDASAVTGIPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMHLVCTQHHAKCCGEHRRNGRHSLCPLCSYLEVAASQGGGMTAVSTPYPVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 42 - DNA, 1041 bp, Cricetulus griseus
atggagaacttccaaaaggtggagaagatcggagagggcacgtacggagtggtgtacaaagccaaaaacaagttgacgggagaagtggtggcgcttaagaaaatccggctcgacactgaaactgaaggtgtgcccagtactgccatccgagagatctctctgcttaaggaacttaatcaccctaatattgtcaagctgctagatgtcatccacacggaaaataagctttacttggtttttgaatttctgcaccaggacctcaagaaatttatggatgcctctgctgtcactggcattcctcttcccctcatcaagagctatttgttccagctgctccagggcctagcattctgccattctcaccgggtcctgcaccgagaccttaagccccagaatctgcttatcaatgcagaggggtccatcaagttggcagactttggactagcaagagcctttggagtccctgttcgtacttatactcatgaggtggtgaccctgtggtaccgagcacctgaaatccttctaggctgcaagtactactccacagccgtggatatctggagtctgggctgcatctttgctgaaatgcacctagtgtgtacccagcaccatgctaagtgctgtggggaacacagaagaaatggaagacacagtctctgcccgctgtgctcctatctagaagtggctgcgtcacaaggaggggggatgaccgcagtgtctaccccataccccgtgacccggcgggccctgtttcccggagattctgagatcgaccaactcttccggatcttccgaactctggggacccctgatgaggtggtttggccgggagtcacttctatgcctgattataagccgagtttcccgaagtgggctcggcaagattttagcaaagttgtgcctccactggatgaagatgggcggagcttgttatcgcaaatgctgcactatgaccccaacaagcggatttcagccaaagcagccctggcccaccctttcttccaggatgtgactaagccagtaccccaccttcggctgtga

SEQ ID NO: 43 - protein, 298 aa, Rattus Norvegicus
MENFQKVEKIGEGTYGVVYKAKNKLTGEVVALKKIRLDTETEGVPSTAIREISLLKELNHPNIVKLLDVIHTENKLYLVFEFLHQDLKKFMDASALTGLPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 44 - DNA, 1400 bp, Rattus Norvegicus
ccggttttttaaaagagactttcttggccaggtggaatttaatctcacccaagggatggtgaaaattgtgagggaaagggctgccgtgctcacccgggcccccaggtcctccgagtgtccggcccgctttccgtcagggttcccgggccccgctcccgggggcctgagccgccctactagcgctccatggagaacttccaaaaggtggagaagattggagagggcacgtacggagtggtgtacaaagccaaaaacaagttgacgggagaagttgtggcgcttaagaaaatccggctcgacactgagactgaaggtgtgcccagtactgccatccgagagatctccctccttaaggagctcaatcaccctaacatcgtcaagctgctggatgtcatccacacagaaaataagctttatctggtctttgaattcctgcaccaggacctcaagaagtttatggatgcttctgctctcactggacttcctcttcccctcatcaagagctatctgttccagctgctccagggcctagcattctgccattctcaccgtgtcctgcaccgagaccttaagccccagaacctgcttatcaacgcagaggggtccatcaagctggctgactttggactagcaagagcctttggagtccctgtccgtacttacactcatgaggtggtgaccctctggtaccgagcaccggagattcttctgggctgcaagtactactccacagccgtggacatctggagcctgggctgcatctttgccgaaatggtgacccgcagggccctattccctggagactctgagattgaccaactcttccggatctttcggactctggggaccccagatgaggtggtttggccaggagttacttctatgcctgattataagccaagtttccccaagtgggctcggcaggattttagcaaggttgtgcctcccctggatgaagacggacggagcttgttatctcaaatgctgcactatgaccccaacaagcggatttcagccaaagcagccctggctcaccctttcttccaggatgtgactaaaccagtgccccaccttcgactctgatgtccttcccaaagccctcttcacctgtggtctgacctgatcctgggccttttggacacaggtcagccttctgctgttttctggctgtcttagccttttctctccggttctggggattcagaggtgcagagaaggattaggtgaaaagggagaaagttttggcattagatgcacttaacccgacttccagcttctctcctcttcccagtcatcactgagaaggattagtattgaagcaaactgcaagtctccagtgtcagattgcatcactagctgctatagtacggtaggatgcagtctgtgcatgttttaactat

SEQ ID NO: 45 - protein, 346 aa, Rattus Norvegicus
MENFQKVEKIGEGTYGVVYKAKNKLTGEVVALKKIRLDTETEGVPSTAIREISLLKELNHPNIVKLLDVIHTENKLYLVFEFLHQDLKKFMDASALTGLPLPLIKSYLFQLLQGLAFCHSHRVLHRDLKPQNLLINAEGSIKLADFGLARAFGVPVRTYTHEVVTLWYRAPEILLGCKYYSTAVDIWSLGCIFAEMHLVCTQHHAKCCGEHRRNGRHSLCPLCSYLEVAASQGGGMTAVSTPYPVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 46 - DNA, 1544 bp, Rattus Norvegicus
ccggttttttaaaagagactttcttggccaggtggaatttaatctcacccaagggatggtgaaaattgtgagggaaagggctgccgtgctcacccgggcccccaggtcctccgagtgtccggcccgctttccgtcagggttcccgggccccgctcccgggggcctgagccgccctactagcgctccatggagaacttccaaaaggtggagaagattggagagggcacgtacggagtggtgtacaaagccaaaaacaagttgacgggagaagttgtggcgcttaagaaaatccggctcgacactgagactgaaggtgtgcccagtactgccatccgagagatctccctccttaaggagctcaatcaccctaacatcgtcaagctgctggatgtcatccacacagaaaataagctttatctggtctttgaattcctgcaccaggacctcaagaagtttatggatgcttctgctctcactggacttcctcttcccctcatcaagagctatctgttccagctgctccagggcctagcattctgccattctcaccgtgtcctgcaccgagaccttaagccccagaacctgcttatcaacgcagaggggtccatcaagctggctgactttggactagcaagagcctttggagtccctgtccgtacttacactcatgaggtggtgaccctctggtaccgagcaccggagattcttctgggctgcaagtactactccacagccgtggacatctggagcctgggctgcatctttgccgaaatgcacctagtgtgtacccagcaccatgctaagtgctgtggggagcacagaagaaatggaagacacagtctctgcccgctgtgctcctatctagaagtggctgcatcacaaggaggggggatgaccgcagtgtctaccccataccccgtgacccgcagggccctattccctggagactctgagattgaccaactcttccggatctttcggactctggggaccccagatgaggtggtttggccaggagttacttctatgcctgattataagccaagtttccccaagtgggctcggcaggattttagcaaggttgtgcctcccctggatgaagacggacggagcttgttatctcaaatgctgcactatgaccccaacaagcggatttcagccaaagcagccctggctcaccctttcttccaggatgtgactaaaccagtgccccaccttcgactctgatgtccttcccaaagccctcttcacctgtggtctgacctgatcctgggccttttggacacaggtcagccttctgctgttttctggctgtcttagccttttctctccggttctggggattcagaggtgcagagaaggattaggtgaaaagggagaaagttttggcattagatgcacttaacccgacttccagcttctctcctcttcccagtcatcactgagaaggattagtattgaagcaaactgcaagtctccagtgtcagattgcatcactagctgctatagtacggtaggatgcagtctgtgcatgttttaactat

SEQ ID NO: 47 - protein, 298 aa, Carassius auratus
MESFQKVEKIGEGTYGVVYKAKNKVTGETVALKKIRLDTETEGVPSTAIREISLLKELNHPNIVKLHDVIHTENKLYLVFEFLHQDLKRFMDSSTVTGISLPLVKSYLFQLLQGLAFCHSHRVLHRDLKPQNLLINAQGEIKLADFGLARAFGVPVRTYTHEVVTLWYRAPEILLGCKYYSTAVDIWSLGCIFAEMITRKALFPGDSEIDQLFRIFRTLGTPDESIWPGVTSMPDYKPSFPKWARQDLSKVVPPLDEDGRDLLGQMLIYDPNKRISAKNALVHRFFRDVTMPVPPLRL

SEQ ID NO: 48 - DNA, 1205 bp, Carassius auratus
ctaaaattactctctctatacagccgctgttttaagtagctttagagtggcagttttccccggaaaaggggcagtttgacatggagtcctttcagaaagtcgagaagattggagaaggaacatacggggttgtttataaagccaagaataaagtcaccggagagacagttgcactaaagaaaattcgattagacacagagactgaaggtgttcccagcactgccatacgtgagatctctctgctaaaagagctcaatcacccaaacatagtcaagttgcatgatgtgatacacacagaaaataagctttacttggtctttgaatttcttcaccaagacctgaagaggtttatggactcgtccactgtcactggcatatccttgccactcgtgaagagttacctgttccagttgctccagggactggccttctgtcactctcatcgtgttcttcatagggatcttaaaccccagaatctcctgatcaacgctcagggtgagatcaaactggctgactttggtctggccagagcgtttggtgtacctgtgcggacttacacacacgaggttgtaactttgtggtacagagctccagagattctcctgggatgtaaatattattctacagcggttgacatctggagtttgggctgtatctttgcagaaatgatcactcggaaggctttgtttcctggagactctgaaatagaccagctctttcggatatttcggacacttggcactccggatgaatctatatggcctggagtcacctcaatgccagactacaaaccctcctttcccaagtgggcacgacaggacctgtctaaagtggtgccacccctggatgaagatggcagagaccttcttgggcaaatgttgatctatgatcctaataagaggatctcagcaaagaacgcccttgttcatcggttcttccgtgatgtcaccatgccagtgccccccttgcgcctctgaagtcatccgtgatgatccttcacagcactcgagtttggatccactttctgagagttattccccatttctttggaaattgatcagttttatactatgactttttttctccattattcaaagataatatggttctaattaatgattttaaaccaaatttctgatggattcataaaatagtggtctctgcaacttaataaacttgttacattgttaaaaaaaaaaaaaaaa

SEQ ID NO: 49 - protein, 299 aa, Sphaerechinus granularis
MNNFEKIEKIGEGTYGVVYKAKDLKSGKTVALKKIRLDTESEGVPSTAIREIALLKELDHKNIVKLHDVVHSDKKLYLVFEFMNQDLKKYMDIAPPSGLPTALVKSYLQQLLHGIAFCHAHRVLHRDLKPQNLLIDADGHIKLADFGLARAFGVPVRTYTHEVVTLWYRAPEILLGCRFYSTAVDIWSIGCIFVEMITRRALFPGDSEIDQLFRIFRTMGTPDEKLWPGVTSLPDYKTSFPRWSPQDFNKIVPMLSKDGKDLLKCMLCYEPDKRISAKTALSHPYFKDVKLVPPPHLPT

SEQ ID NO: 50 - DNA, 1410 bp, Sphaerechinus granularis
attatcactacggtatggcgttagaagaccgtcaatatgaataactttgaaaagattgagaagattggcgaaggcacttatggagtggtttacaaggcaaaagacctaaaatctggaaaaaccgtcgcactgaagaaaatacgattggatacagaatcggaaggtgtcccaagtactgccatcagagaaatcgcactcttgaaggaactggaccacaagaatatagtaaaattacatgatgtggtgcacagtgataagaagctgtatcttgtctttgagttcatgaaccaggatctcaagaaatacatggatattgcacctccgtctggtctaccaacagctttagtcaagagttatcttcagcaacttctacatggtatagcattctgccatgcacaccgtgtccttcacagagacctcaagcctcagaatctgctcattgacgcagatggccacatcaaacttgctgattttggactagcaagagcatttggggttcctgtcaggacatacactcatgaggttgtcacattatggtatcgtgctcccgaaatcttactaggctgcaggttttactctacagcggtggacatctggagtataggatgtatctttgtagagatgataacaagaagagctctattccctggtgattctgaaattgaccagctcttcaggatattccgaacaatgggcactccagatgaaaaactctggcctggggtcacctctctacccgactacaaaaccagtttcccaagatggtcacctcaggatttcaacaagatagtccccatgctcagtaaagatggcaaggacttactcaagtgtatgctgtgctacgaaccagacaagaggatatcagccaagacagctctctcacatccatatttcaaggatgttaaactggtgccgcctcctcatcttccaacatgatcaacttgcaccatgcaaagccttcttatagataaaatgtatgtgttgaagagagacattaccttatttattacacatctttgtgtatattatgtataaatagtgagaaagggtttgtgttttgtttatggttattttgtggtgtacatgacaatattccacttagcttactagtaatctgattgctctaggactgtaataattgaacattatacaacaattttgtgccttaaaaattatgtatgtggggtgacaacgcattatatctcaaatctattgacttttcaggaggataaaaagacatggtcattatgaaagtactctctgtcatgatatccttcatttacagagcaatagtgacttgaactctgcagtgcgtattttgaggaataagggatttgacaagacaaattttgtctcgagctattaagcatgaaaaaccccaccaggttgagctactgggtttcacacctg

SEQ ID NO: 51 - protein, 297 aa, Xenopus laevis
MENFQKVEKIGEGTYGVVYKARNRETGEIVALKKIRLDTETEGVPSTAIREISLLKELNHPNIVKLLDVIHTENKLYLVFEFLNQDLKKFMDGSNISGISLALVKSYLFQLLQGLAFCHSHRVLHRDLKPQNLLINSDGAIKLADFGLARAFGVPVRTFTHEVVTLWYRAPEILLGCKFYSTAVDIWSLGCIFAEMITRRALFPGDSEIDQLFRIFRTLGTPDEVSWPGVTTMPDYKSTFPKWIRQDFSKVVPPLDEDGRDLLAQMLQYDSNKRISAKVALTHPFFRDVSRPTPHLI

SEQ ID NO: 52 - protein, 1131 aa, Xenopus laevis
IPRGTKPLPSPVSFLQLSTQREVVARVIQRICEKKRKNVLAFGYGLVDEKNSLNIRLTPNICNYFPNPTTTTISTSILWETLLTRVGDDVMMYWLEQCSIFVFVPPRCCYQITGQPIYTLPSDDVFLFQSQSFTQSNVLLRYIKRNVFHLRKKYLKPKHSMTSRMLTWRRNKSPSGLLIRSKTSMAVTTEIHSKRKLCSKDICVIPDKKRRDNLDKDDTVDHFDLPMCRSVSYLSNMYPKTNVQVTGLITSGYKKTKTFQCQKPVSCEQKKTTAFYSVAGDCNLSLKDNVNKLITNASVPTAQSRLSFSNIFIDFGRTLYLSISYKKGFSESFILNSLDSTPSGSQKLVETIFLSNFLAEQNFDQPKRDENCRYKLPKRYWKMKPHFQELIQNHKKFPYLVYLNKHCPVRSSMACSEKRSLQKNRIENDGKQLKHFTTKANLLSLLKQHSSIWQVYMFVRECLNNVVPDIMWGSSHNKCRFFRNVKSFLFFSGKFGKISLSELMWSMRVEDCSWIRLQKSDHFVPASEHLLREKILAKFVFWLMDTYVIQLLKSFFYVTETMFQKHRLLFYRKSVWKKLQNIGLRKHLEKVKLRSLSSDELENMQQWKNVPLVSRLRFIPKTNGLRPISKISSTLSSQQSKENQEKKIHHFSSQIRNLFSVLNYEWNRNCSLIGSSVFGMDDIYKKWKKFVLDFEKPQVEKLQFYFVKTDVKGAYDTIPHSKLDEVISKVINPNANEVYCIRRYATVSVDPTGRIIKSFKRHVSELADVLPNMKQFVSNQQEKNLLRNTILVEQNLLLNESSVKLLAVFQQIIRSHILRIKDRYYMQCCGIPQGSMLSTILCSLCYGDMENAMLGGIQKNGVLMRLIDDFLLVTPHLDQAKTFLRTLAEGIPQYGCSISPQKTVVNFPVDDIPECSEVEQLPSHCLFRWCGLLLDTQTLDVYYDYSSYACTSIRSSMTFCHSSAAGKYMKQKLIRVLRLKCHSLFLDLKVNSLRTVCINTYKIFLLQAYRFHACVVQLPFGQRVMNNPPFFLTVISDMAPCFYTTFKAKNKDLTRGYKDVSCQFNFEAVQWLSYQAFLTKLHNHKVLYKCLIGPLQNCKMQLSRRLSQDTIELLKSVTDSSLHKDFSCIMD

SEQ ID NO: 53 - protein, 1128 aa, Mesocricetus auratus
MPRAPRCRAVRALLRSQYRQVWPLATFVRRLGPEGRQLVQPGDPKVFRTLVARCLVCVPWDSQPPPADLSFHQVSSLKELVARVVQRLCERGERNVLTFGFALLNGAQGGPPMTFTTSVRSYLPNSVTESLRVSGAWMLLLNRVGDDLLVYLLARCALYLLVPPSCAYQVCGSPLYQICATAETWPSVSRIYRPTRPVGRNFTHLGSTHRVRNSSHQEAWKPPPLPSREAKRSLSITNRSVPPSKKARCDLAPRLEKGPYRQAVPTPSDKTWVPNPAKSHAVPISRTTKEDLSSGVKAPGLSRSGSVCYKHKPSSTSLQSPLCQNAFQLRPYTETKRFLYSREGGRERLNPSFLLNNLQPSLTGARRLVEILFLGMRPRTSGPLCGRRRLSKRYWQMRPLFQQLLVNHARCPYVRLLRSHCRFRTAAHQVAGALNTTSPQRLMNLLRLHSSPWQVYGFLQACVGKLVPPGLWGSRHNQRRFFKNVKRFISLGKYDKLSLQELTWKMKVQDCRWLRSSPGNNCVPAAEHRTRERILAVFLFWLMDAYVVELLRSFFYVTETTFQKNRLFFYRKSMWRRLQSIGVRHHLERVRLQELSQEEVRQRQEAWPAMPICRLRFIPKPSGLRPIVNMSYMGTRAFDKGKQAQHFTQCLKTLFSVLNYELTKHTNLLGASVLGLNDIYRTWRTFVLRVRTLDPAPRMYFVKADVTGAYDAIPQDKLVEVIANMIRHPDNSYCIHQYAVVQRDRQGQIHKSFRRQVSTLSDLQPHMGQFLKHLQDSDTSALRNSVVIEQSLSLNEASSSLFDFFLRFVRNSVVKIGGRCYVQCQGIPQGSSLSTLLCSLCFGDMENKLFAEVQQDGLLLRFVDDFLLVTPHLVQAEAFLRALVRGIPEYGCMINLQKTVVNFPVDAGTLDGTAPHQLPAHCLFPWCGLLLDTQTLEVLCDYTGYARTSIKASLTFQRTFKAGRNMRQKLLAVLRLKCHSLFLDLQMNSLQTVCINVYKIFLLQAYRFHACALQLPFDQHVRKNPAFFLSIISNIASCCYSILKVKNAGMTLKAKGASGSFPPEAARWLCYQAFLLKLAGHSVTYKCLLGPLRTAQKQLCRKLPRATMAILETAADPALSTDFQTILD

SEQ ID NO: 54 - protein, 2629 aa, Rattus Norvegicus
MEKLCGYVPVHPDILSLKNRCLTMLSDIQPLEKIHGQRSVNPDILSLENRCLTLLPDLQPMEKIHGQRSVHPDILSSENRCLTLLPDLQSLEKLCGHMSSHPDVLSLENRCLATLPTVKRTVSSGPLLQCLHRSHTAQADLRDPNFRNCLFPEPPTIEAPCFLKELDLPTGPRALKSMSATARVQEVALGQRCVSEGKELQEEKESAEVPMPLYSLSLGGEEEEVVGAPVLKLTSGDSDSHPETTDQILQEKKMALLTLLCSAMASSVNVKDASDPTRASIHEVCSALAPLEPEFILKASLYARQQLNLRDIANIVLAVAALLPACRPHVRRYYSAIVHLPSDWIQVAEFYQSLAEGDEKKLVPLPACLRAAMTDKFAQFDEYQLAKYNPRKHRSKTRSRQPPRPQRTKPPFSESGKCFPKSVWPLKNEQISFEAAYNAVSEKKRLPRFTLKKLVEQLHIHEPAQHVQALLGYRYPSTLELFSRSHLPGPWDSSRAGQRMKLQRPETWERELSLRGNRASVWEELIDNGKLPFMAMLRNLCNLLRTGISAHHHELVLQRLQHEKSVIHSRQFPFRFLNAHDSLDRLEAQLRSKASPFPSNTTLMKRIMIRNSKKIKRPANPRYLCTLTQRQLRAAMAIPVMYEHLKREKLRLHKARQWTCDLELLERYRQALETAVNISVKHNLPPLPGRTLLVYLTDANANRLCPKSHLQGPPLNYVLLLIGMMMARAEQTTVWLCGTGTVKTPVLTADEGILKTAIKLQAQVQELEENDEWPLETFEKYLLSLAVRRTPIDRVILFGQRMDTELLNVAKQIIWQHVNSKCLFVSVLLRKMQYMSPNLNPNDVTLSGCTDGILKFIAEHGASRLLEHVGQLDKIFKIPPPPGKTKVSPLRPLEENNPGPFVPISQHGWRNIRLFISSTFRDMHGERDLLMRSVLPALQARAFPHRISLHAIDLRWGITEEETRRNRQLEVCLGEVENSQLFVGILGSRYGYTPPSYDLPDHPHFHWTQRYPSGRSVTEMEVMQFLNRGQRSEPSDQALIYFRDPGFLSSVPDVWKPDFISESEEAAHRVSELKRFLQEQKEVTCRRYSCEWGGVAAGRPYTGGLEEFGQLVLQDVWSVIQKRYLQPGAQLEQPGSISEEDLIQASFQQLKSPPSPARPRLLQDTVQQLMLPHGRLSLVIGQAGQGKTAFLASLVSALKVPDQPNVAPFVFFHFSAARPDQCLAFNLLRRLCTHLHQKLGEPSALPSTYRGLVWELQQKLLLKSAQWLQPGQTLVLIIDGADKLVDHNGQLISDWIPKSLPRRVHLVLSVSSDSGLGETLQQSQSAYVVALGSLVPSSRAQLVREELALYGKRLEESPFNNQMRLLLAKQGSSLPLYLHLVTDYLRLFTLYEQVSERLRTLPATLPLLLQHILSTLEQEHGHNVLPQALTALEVTHSGLTVDQLHAVLSTWLTLPKETKSWEEAVAASHSGNLYPLAPFAYLVQSLRSLLGEGPVERPGARLCLSDGPLRTAVKRRYGKRLGLEKTAHVLIAAHLWKMCDPDASGTFRSCPPEALKDLPYHLLQSGNHGLLAKFLTNLHVVAAYLEVGLVPDLLEAYELYASSKPEVNQKLPEADVAVFHNFLKQQASLLTQYPLLLLQQAASQPEESPVCCQAPLLTQRWHNQCILKWINKPQTLKGQQSLSLPISSSPTAVAFSPNGQRAAVGTAGGTIYLLNLRTWQEEKALVSGCDGISSFAFLSDTALFLTTFDGLLELWDLQHGCWVFQTKAHQYQITGCCLSPDRRLLATVCLGGYVKLWDTVQGQLAFQYTHPKSLNCITFHPEGQVVATGNWSGIVTFFQADGLKVTKELGGPGPSVRTLAFSAPGKVVALGRIDGTVELWAWQEGTRLAAFPAQCGGVSTVLFLHAGGRFLTAGEDGKAQLWSGFLGRPRGCLGSLYLSPALSVALNPDGDQVAVGYRGDGIKIYRISSGPQEAQCQELNVAVSALVWLSPSVLVSGAEDGSLHGWMLRRNSLQSLWLSSVCQKPVLGLAASQEFLASASEDFTVRLWPRQLLTQPHAVEELPCAAELRGHEGPVCCCSFSPDGRILATAGRDRNLLCWDVKVAQAPLLIHTFSSCHRDWITGCTWTKDNILISCSSDGSVGLWNPEAGQQLGQFPGHQSAVSAVVAVEEHIVSVSRDGTLKVWDRQGVELTSIPAHSGPISQCAAALEPRPAGQPGSELMVVTVGLDGATKLWHPLLVCQIHTLQGHSGPVTAAAASEASGLLLTSDNSSVRLWQIPKEADDTCKPRSSAVITAVAWAPDGSLVVSGNEAGELTLWQKAQAVATARAPGRVSDLIWCSANAFFVLSANENVSEWQVELRKGSTCTNFRLYLKRVLQEDLGVLTGMALAPDGQSLILMKEDVELLQMKPGSTPSSICRRYAVHSSILCTSKDYGLFYLQQGNSGSLSILEQEESGKFEKTLDFNLNLNNPNGSPVSITQAEPESGSSLLCATSDGMLWNLSECTPEGEWVVDNIWQKKSRNPKSRTPGTDSSPGLFCMDSWVEPTHLKARQCKKIHLGSVTALHVLPGLLVTASEDRDVKLWERPSMQLLGLFRCEGPVSCLEPWMEPSSPLQLAVGDAQGNLYFLSWE

SEQ ID NO: 55 - protein, 2629 aa, Rattus Norvegicus; identical to SEQ ID NO: 54

SEQ ID NO: 56 - DNA, 4425 bp, Cryptosporidium parvum
atgagtgtgttgaagggtgctttagcagtgttgatgaagcaagaggttctgacgctgggagagtacctggagcagaaaagaaagattagagtattcaaagaagaaggattgtacaatgattcctcaggggagttaaggaggctacttgaagagactttgatactggaagatccttctataccaaaagctttaaaggagggatttcttggcttaagggaacttacatttaattccttagacaatagtcttgatattaacggagacaggaggaagttgttgggtataaatatcgaaaaattgctcgagtggtcatcgatactaggtcagaactactttagctcacctagaaatcagtcgtctatgtcttcaggaaagaggtcaagaaataccgaatataatgagacattgtctccttcctttattacagaccaaattaagaatataattccagattctgaaaaggattctctgacctatcaagtacaagactttgagctcctactccgtaagcactttccaaggattaggatctttcattcagggagttattcaggattaagaactagcttcgacactataaagcccaagaaatactcattctgtaattctactttatacaagtttcaaaacctccatgcttggaatactcttctttccaaaataggtcctattgagattctcttcttatttgtttgctgtattatctttagaatacttggtgatcactctgaaattctcattcaacaagctggtagaatgttaactaatgacttcctggaagagcttgctagactttatgagacagggccaaaaaagaccaattcatttgtatcatcatcttctttaactccaattcttacaattgaggaaaaagaagaagttaagcctttagatgaaactccaagacaagtaaacaaaagatcaggaggaattattaaagattcaagactttctaaaatctacaaaattgatattccatatcgctcaacatttctttattgtgatcatttctcaaaaagaggaggtcttccttgtttatctgttctaagacttttacctccaaatttattaggagcaagaacactacttagatttattattcaaactgatcatatattcaaagatcacaacagacaaagtttgattggattacttggaagttatgaaatgacaaaattctcccgtgttaaatgtaaattggcttctgctatgatggaagaattccaaaatttattgttaaatattagaaatacgtctccagttaactttctagatcaaatatgtccaatagaaccaattaaagattcagatttacaaaacttcaataaattacctattccttgttttgaaacaagttctactagagtagtaaattttttaagattatacttgattaaagtattaccaaagaatatacttggtacctttaaaaactttaagacttttattaataaaaagatcccaataattgtgaatcttcatattagagaaacttttaagattcaacatgcaatgaatggaattgaggtttcaaattgggtaaatagaatacttgaagaaaaaagttatcaatttattaaagaatcaaaaaatttaattaattctaatcaaagatctaaaaaggtaccgaataataagacaagatctagtattaaaaaaagtttaattagccttggaaaaacatatctagctaggaacatttattttttaatagtatatttggtatttccaatattaagaagacatttctatgcaacagaaattgagggtagtaataaagtaagatattttagacatccagtttggataaagatcgtgagacaagctgataaatggtatctagaaagtatattaaaaggaattcagtcaaaagactatataatgaatttatcttcaattcaagaaatttcaaaattaattgataagaactctgagttttctgaaaatatcccaaaaataagatgggtaccgaaatctaaaggtttaagaccattaataaatttatcaaaagttggatctggacaaattcttcaacaaaatcttgaaagagatcacacttgtgaagaaaagaagaagattggtttttgtgattgtaattcagtttggactggtggagatcttccaacaagttggattaataacaattactattataactactttaatagtaatggtaataacggtattgtaggaaataatcataattatggtcatattaataatataggaggaaagaattttacatcattttcaagccaaaattgtcagattatgacaccaccaggaagattaggagtgggattaggatttggtaattataatataggaattggaataaattcaggatcatcggtacctactactccaattaataaggtaatgagacgtccttcaagtaatagaattatagatataagacgtccatcaacaaataatatgttgttttatccttccaagattttaagatcttttctattaagaatgacaggaaagaatcatcttggtgcttctcttgtacagtttggagatatttataagattataaagaattggtggataaagaatagaagagacaaacaacaatttcaaatgaatgaagatttttatcaaaatcaaggtaaaaaaatgtcccatgtaaagatttatattattaaagcagatttagtgaactgttttgaaaatataaataagtccaagatttttgagtttttggatgtaatttctcttccaaatgaaatttcccttttgagcttatattcaagggctttaagtaaaacaacaattattccgccttttgataatatgaatagagactcttaccaagatgagcttgggagatcatgtattattacttcaaaaggaaaactaaatatggtacctatttttgagaaagatcatgatgaagatcaaaaagtcgtcaaatcaaaaattaagaggattataggaccagatttagatgaacttctttggaatcttaaaaattcttgtttatcaaacaaggatttgagtattttgggacaaaaaaaagcagaaatctttacttttttaaattcaagacgtgtgataaactggaaattagtcaaggagatcattaagatccaccttaacacaagcttttttcgacttaggacatgttcaaagtctttaagactaataaaatctaaacaacttggaaattctatcaaatttggaaaaagatttctaagcttatttaagcaagattttggaattccacaaggatcaagtatttcttacattctttgttgtctctattataatttcttagatctaaatccagaaatccagaatcttttgggacattcatttttttcctcctcttacatttctttatcatttctgaatccaattaaacaagaaaaagtacaattacttgatccaactgaaaaagacttattttcacaacatgatcttgagacaggattatcccgttacccagaatttaaagaaaatgagataaatatttataatactagtacaagtaaaaaaagaagactagaaatatcagaatataatattaatgctaaaagtgttttactgcaaccttccgaagagtttaataataatatcaacaaccagaattgccaaatccgtgccaatctcaaaagttatgatcaagaaagtccttctcagagcaataagcaagaaagtctcttattgagatgggtagacgattttctttttctcacatccgatttggagtccgccaaaaagttcttaaagttactttatatacaaaaactatggggatcgaacgtttccaaggacaaaatcaactctaactttccttggattgaccacaataatgagatcatcatccttgaggaggatgatgttttactatcttcgccttcctcttcctgttcatcatcttgttcctcttcatcttcctcttcatcttcttcgtcgtcttcctcatcgtcctcttcttccttcccaccttcgacagagtgcacaagcagtccagtggaaaagagaataaataccaacaaaagtgaaaaggactgccaaaatgaaaaagaaatgattaacaaggctaagatagcctcaaaacagttccataaacaagtatcatggacaggaatgaagttttccagtgagtacagctatttgaattgcatggtttccccatggaagaatttggaattcatatgtgttatggatactgtaaccttgacaactaagcaccaatttacaagtaattattcaaattttcacaggtttaagtcgacgacgatatctgaaaaccttcaaaagtcaaattatatgtggtctgttttgggtataaaattaataagatactttgattttagaatcaagaatggattgctatatgattgtaaaatcaattctttatttacggtaagttttaaatataattcaacaaataaccaaccaattaattaa

SEQ ID NO: 57 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 58 - DNA, 4425 bp, Cryptosporidium parvum; identical to SEQ ID NO: 56

SEQ ID NO: 59 - protein, 960 aa, Giardia intestinalis
MDFPLSLLDANVSKAIFILLQGEPEQASLWSLILQLRTPGQGSSFASCASLIYKDLKGLSSWITEAAFIPFLRRLGFCSLYLSYGHDVLLELFKYPIVLTSTVSGKRVLLSEPQMPLAFPPCLHDKSVSSMVRASTVQYASNCLEEFISMIQEHPMKLRFIFFCKTRPYIRAFHSLALLGSATAIWHLSLLPSQCLTAFYQANPLVTFKFPYNAFSTRSFSSLLQLCTSRGRLMPNNAINSNAKARITRLISIFRSKMKRLSHNLTFLKDKKPQSLLQSFGFDTHFRVFLDETGTLRHAPLPLSVHKITLYLMLYCKSISAFSILGKRNARTFHSHLFRFISQPMNGFMTLQELTSSISTKEFLLPATTVSKAEGEVRHHLLAYFVVYLMDFVILPALYQSFSAVSMHTELSNRIETANSMVPALLSRSLWVSYKNYLIKSSARLEIAATYEFVCSLSNLHSIPLTNEDAVNRIFWKQVSPGRLSFVYKSNGRVRPLCNFSFCSRRHRVSLNSFLRSALQVIIFELERPRNRYLQAHLAKNLGHIAVDYAAWLDATYTENPEAQIYMVTTDMITAFESVSVPRLLYYISHYIVTGNAYVVLTYKEIAPSKMPKIRTVALPCQHNGCVSMDSISRYLLCKETPKKQPNSMLCPMHARIYKREDIIKMLELHLLNPLVIHEGGVYRLTSGIPQGSVVSTVLFNCYSSLLHVQLLNTGIVSSRLFFYRTFVDDWLLLTTSIALLDAYIEYLNIMRDHGAYFRLSCFTKPHKNSPFAPIATLDCSREPIPKKSLYTPNLNQDMYKLDVPRYCGYIFLENVAIIDVLKWFSSTRTNGSPSYPMIFGSGISVMQRPRLVFKKRIRDLRCFIERSIQAPDPRIKVDGSILYAYIRAVTFCILCYTRNIRYSESLRTLLRSTFCSLKATVWRHSTSRAIFIHVSKRIILHVYGHNPRIRGLLLGMLSR

SEQ ID NO: 60 - DNA, 2883 bp, Giardia intestinalis
atggattttccactgtctcttttggacgcaaatgtcagcaaggctattttcatcttgcttcaaggggagcccgagcaagcctccctatggtcacttatccttcaactgcgaacccccggccaggggtccagcttcgcttcttgcgcatcgctgatttacaaagacctcaagggacttagctcctggattaccgaggctgcttttattccctttctcagacgtttgggattttgttcattgtatctaagttatgggcacgatgttctgctcgagcttttcaaataccctatcgttctcacttcgactgtttcagggaagcgggtgctcctttcagagccccagatgcctcttgctttcccaccttgcctccatgacaaatctgtatcatctatggttcgagcttccactgtgcagtatgcttctaactgtcttgaggaattcatttctatgattcaagagcaccccatgaagctacgattcatattcttctgtaagacccggccctacatccgagcattccacagcttggcattactaggatcagcaacggccatctggcacctttctcttcttccttcacagtgccttactgccttttatcaagccaatccattagtcacatttaagttcccgtacaatgccttttccacgcgctcgttttcatctttgctacagctatgcacatcccgtggtcggctgatgccgaacaatgctattaattcaaatgctaaagctagaataacaagactcatttccatttttcgtagtaagatgaaacggttaagccacaatcttacgtttctgaaagacaagaaaccccaatctcttttgcaatcatttggctttgatacgcattttagggtattcttagatgaaacaggcacccttcgtcacgcccctctgcccttatctgtgcacaaaataactctttatctcatgttatattgcaagtccatcagcgcattttctattcttggcaaaaggaacgctcgaacatttcactcacatctctttcgctttatttctcagccaatgaacgggtttatgaccttgcaagagctcacctctagtatctctacaaaagaattccttctcccagcaaccacagtcagcaaggccgagggagaggtgcgacatcaccttttagcgtacttcgttgtctatctcatggacttcgtcatacttcccgccctctatcagtcgttttctgctgtttctatgcacacggagctttccaatcggatcgagacggccaactctatggttcctgcattgctttcccgctcactttgggtcagctacaagaattacttgattaaatcgtctgctcgtctagaaatagcggccacttatgaatttgtctgctcattatccaatttgcactctatccctctaaccaacgaagatgctgtgaaccgtatattttggaagcaagtctctcccggtaggctatcgtttgtctataaatctaatggaagggtgcggcccctatgtaactttagtttttgtagccgacgacacagggtctcacttaattcctttcttcgaagcgcgctccaggtcatcatatttgagctagagcgcccacgcaataggtacttgcaagcacatctagccaagaatctaggccacatagcagtggactatgctgcttggctcgatgcgacgtacacagaaaaccccgaggcgcagatatacatggtcacaaccgatatgataactgcttttgaaagcgtctccgttcctcgcttactctactacatatcccattacatcgttactgggaatgcttacgttgtcttgacttacaaggagattgctcccagtaagatgcctaagattaggaccgttgcgctgccctgccagcataatgggtgtgtatccatggattctatttcccgctaccttctctgcaaggagacccctaaaaaacagcctaattctatgctgtgtcccatgcatgcacgaatctataagcgagaggacattatcaagatgctagagcttcatcttctcaatcccctcgttattcacgaaggaggagtataccgtctgacatctgggataccccagggcagtgtcgtaagcactgtccttttcaattgctattcgtctctcctgcacgtgcaactactaaatacagggattgtttcgtcccgtctcttcttttacaggacctttgttgacgactggctgctattaactacgtccatagcattacttgacgcatatatcgagtacctgaacatcatgcgtgatcatggagcctacttcagattgtcctgcttcacgaagccacacaaaaacagcccattcgctcccatagctacattagactgttctagagaacccataccaaagaaaagtctttacacacccaatctcaatcaagatatgtacaaactagacgttccacgatattgtggatatatatttcttgaaaatgttgccataatagacgtactgaagtggttttcatcaacgcgaaccaatggcagtccgtcctatcccatgatttttggatctgggatatctgtcatgcagagacccaggttggttttcaaaaagagaatccgggatctccgctgtttcattgagagaagcatacaggcacccgatccgcgtataaaagtggacggctctattctttatgcgtatatacgtgctgttacgttttgcatactttgctacacacgaaacataagatactctgagtccttgcgcacccttttaaggagcacattctgttctcttaaggcgaccgtgtggagacattctacatcccgtgctatatttatccacgtatccaaacgcattattctacatgtctatggtcacaatccccgcatcagagggcttcttctgggaatgctaagcaggtag

SEQ ID NO: 61 - protein, 2629 aa, Mus musculus; identical to SEQ ID NO: 54

SEQ ID NO: 62 - DNA, 1255 bp, Mus musculus
taacaacgtgcaagtactatcacagtgccaaactagcagagctgccatctaaggtcgaggtcgccgctttggctgtgtgcacaggcaagcgccctcacccaatggccctggccttgctatgggtgcgtgagttgagatgatgctctggactctgaggtgaaggccactggaacagtgaaaaaagctaacgcagggcttttacctagtccccttcctttggtggtgggtgtttacggaacatatttgggatctgagtgtatggtcgcaccacaataaagccttaacctatatagtagaatttcagctgtaatcattaagaactgagattgccaccacccacctcactgtctgtgtcaaccacagcaggctggagcagtcagctcaggaacaggcaaaaccttaggtccctccgcctacctaaccttcaatacatcaaggataggcttctttgcttgcccaaacctcgccccagtctagaccacctggggattcccagctcagggcgaaaaggaagcccgagaagcattctgtagagggaaatcctgcatgagtgcgccccctttcgttactccaacacatccagcaaccactgaacttggccggggaacacacctggtcctcatgcaccagcattgtgaccatcaacggaaaagtactattgctgcgaccccgccccttccgatacaacgcttggtccgcctgaatcccgccccttcctccgttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgggcctccaggcgggatccccatgggtcaggggcggaaagccgggaggacatgggataatgcgtctagctcatgtgtcaagaccctcttctccttaccaggtgtcatccctgaaagagctggtggccagggttgtgcagagactctgcgagcgcaacgagagaaacgtgctggcttttggctttgagctgcttaa

SEQ ID NO: 63 - protein, 867 aa, Candida albicans
MTVKVNEKKTLLQYVLDNTSNEVPLLPSLKEYMETVLVYQSIKRPLPAIRPQESFDEFMKELVTRLVMEKSNNVIAYGYKTSAMESRSIFTTFHSSGNFILTHITSHNWSTIFSLLGPKKFLELLVNNKGFVSKVNGESVQIFGDVNSHRKAVVVSKYITKFNVLYNSYSRDFSRFEMIRPSIQTILQDILSFSGLNPGRSSKRYRGFKSLLSRIIANDKKCRYDILYAKFIGTSKCNFANVVSNKTEISQVIQFVLLVLGKLLPLDAWGGVSNKKIIKDRVVDFLLLGANEKIHMDDLFRGIRLKDFKWLGRAHQISSKQDFELRTAFLKGYLWWLFEHLLKNILRSFWYITETSSIVSLELNYFPQYLWKELYESWVSKYAKNNLVKMPSKIQREQLPCGKIKLIPKRSSFRVICVPIKRSLKLLNKKLELDTLEKEKREFERYRKEVLSPVGQILRLKLSKLRDTYESYRASVHSSSDVAEKISDYRDSLLTRFGEIPKLFILKFDMKECYDRLSQPVLMKKLEELFENQDNKTSYYVRYYAQLDASHKLKKVKTTIDTQYHNLNILSSSRHLSNCKSLVDKTKTIALQKGNILEVCRSQIYDVVGSVKDARGNLHLYKRKRGVFQGFSLSSIFCDILYSAMVHDCFQFLWKSKQDFLFVRLVDDFLLVTPDSNIYDQVHNILSGKILESYGAFVNKDKTVVVNQTTTKPSIDFVGLEVNTTDLSIKRNSGSISLVTTNFRTFKTLVKYLKTFYQLNLEGFLLDCSFGVLENVLENMGSLLRLVLREFKTKFTSIVKYDTFHCYKFIKFLYDISNYTIVKYVETNSDWDGAPELLNCIKQIIVKEFSSFESYSEIVEWVQTLNI

SEQ ID NO: 64 - DNA, 2714 bp, Candida albicans
cgttgttattcacgcgtatcgtgagatatcatttcaaagaaccacatacatgaccgtcaaagtaaatgagaagaagactttacttcagtatgttctagataatacaagcaatgaagtgccattgctacctagtttgaaagagtacatggagacggtgcttgtataccaatccataaaacggcctctaccagcgattcgaccacaagaatcatttgacgaatttatgaaagagttggtgacccgtttagttatggaaaaatcgaataatgttatagcttatgggtataagacctccgcaatggagagtcgaagtatatttacaacgtttcattcgagtgggaattttattttaactcacattacaagccataactggagtacaatatttctgttactcggacctaaaaaatttctagagctattagttaataataaggggtttgttagtaaggtgaatggtgaatctgtgcaaatattcggtgacgtgaactctcacagaaaggctgtcgtcgtttccaaatacattaccaaattcaatgtgctttacaactcctattccagggacttctcacgctttgagatgataagacccagtattcaaactatattacaggatattctttccttttctggtttgaatcctggaagatcatccaaaagatatcgaggcttcaaaagtttgctctcgagaattattgctaatgataagaaatgtagatacgacattctatatgctaagtttattggtacgtcaaaatgcaattttgctaatgtggtgagtaataagacagaaatatcccaggtaattcaatttgtacttttagtattgggtaaattgttacctttggatgcttggggaggtgtttccaataaaaagattattaaggaccgagtggtagattttttgttacttggggcaaatgaaaagatacatatggatgatttatttagaggaattagactaaaagatttcaagtggttgggcagagctcaccaaatttcttcgaaacaagatttcgagctccgaacagcttttctaaaagggtatctatggtggttgtttgaacatttacttaaaaatattctccgttctttctggtacattactgaaacttcaagtatagtgagtttagagttgaattattttcctcagtatttatggaaagagctatacgagtcatgggtgtctaaatatgcaaagaataatcttgtgaaaatgccatcaaagatccaaagagaacaactaccatgtgggaaaattaaactcatacccaagcgctcgagctttcgtgttatttgtgtacctataaaacgatccttgaaactattgaacaaaaaattggaattggacacattggaaaaggagaaaagggaatttgaaaggtacagaaaagaggttttactgccagtgggacaaatactacgcttgaaattatcgaaactaagagatacatatgaaagctatagggcttcagtacattccagttctgatgtggctgaaaagatactggattatagagactccttgttaaccagatttggcgaaatccctaagcttttcatcttaaagtttgacatgaaagaatgttatgatagactcagccaacctgtattaatgaaaaaactagaggaacttttcgaaaaccaagataataagactctgtattatgttcgatactacgctcagttggacgcgtcacataaattgaaaaaagtgaaaaccactatagatacccagtatcacaatttaaacattttgtcgagctcaaggcatctcagtaattgtaaatctttggtcgataagaccaagacaatagcgttgcaaaaaggtaacattttggaagtttgtcgaagccaaatctacgatgttgttggttcagttaaagatgcacgagggaatttacacctatataaaaggaagaggggcgtgtttcagggattctcattgctgtctatattttgtgacatcctatatagtgcaatggttcatgattgttttcaattcttatggaagtcgaaacaggattttttatttgtacgattggtagatgactttttacttgtaacgcccgattcgaatatttatgatcaagtgcacaatatattatcaggaaaaatacttgagagctatggagcttttgttaataaagataaaacagtcgttgttaatcaaacaaccacgaaaccaagtatagatttcgttgggctcgaagtgaatacaacagatctaagcatcaaaaggaactccggtctgataagtttggttacgacaaacttcagaacattcaagactttagttaagtatttaaagactttctatcaattgaatttggaggggtttctcttggactgttcttttggggtattggaaaacgtgcttgaaaatatgggatccctccttaggttggttttgagggaattcaaaacaaagtttacctccattgtcaaatatgatacatttcattgttacaaatttatcaaatttctatatgacataagtaattacacaatcgttaaatatgttgaaacaaacagcgactgggatggtgcacctgaactattgaattgcattaaacagataattgtcaaggagttttcctcttttgagagttacctggaaatagtcgagtgggtacaaacattgaatatataaatacactgctcatatacccccaaacgagctttttaaattctcgatatctctcaattgtcgc

SEQ ID NO: 65 - protein, 34 aa, Artificial Sequence
  conserved amino acids in cdk4 p16Ink4a binding site
YEPVAEIGVGAYGTVYKARDXXSGXFVALKXVRV

SEQ ID NO: 66 - protein, 11 aa, Artificial Sequence
  protein transport domain
RRRRRRRRRRR

SEQ ID NO: 67 - protein, 11 aa, Artificial Sequence
  protein transport domain
YGRKKRRQRRR

SEQ ID NO: 68 - protein, 34 aa, Artificial Sequence
  Consensus Sequence
YEXVAEIGXGAYGXVXKARDXXXGXFVALKXVRV

SEQ ID NO: 69 - protein, 33 aa, Artificial Sequence
  Consensus Sequence
XXXVXXIGXGXYGXVYKARXXXXGXXVALKXXR

(19) United States
(12) Patent Application Publication
(10) Pub. No.: US 2003/0166270 A1
(43) Pub. Date: Sep. 4, 2003

(54) Compositions and methods for reversibly inducing continual growth in normal cells

(75) Inventors: E. Premkumar Reddy, Villanova, PA (US); Sushil G. Rane, Frederick, MD (US); Richard V. Mettus, Feasterville, PA (US)

Correspondence Address: DRINKER BIDDLE & REATH, ONE LOGAN SQUARE, 18TH AND CHERRY STREETS, PHILADELPHIA, PA, 19103-6996, US

(73) Assignee: Temple University - Of The Commonwealth System of Higher Education, Philadelphia, PA

(21) Appl. No.: 10/295,681

(22) Filed: Nov. 15, 2002

Related U.S. Application Data
(60) Provisional application No. 60/334,760, filed on Nov. 15, 2001.

(51) Int. Cl. C12N 5/06; A61K 38/17

(52) U.S. Cl. 435/325; 514/12

(57) ABSTRACT

A mutant modified cyclin dependent kinase protein, or biologically active fragment, derivative, homolog or analog thereof is provided, which reversibly induces continual growth in cultured cells when administered to the cells exogenously in culture. Methods of reversibly inducing continual growth in cultured cells, and methods of screening cancer-causing agents with the continual growth-induced cells, are also provided.

### CROSS REFERENCE TO RELATED APPLICATION

This application claims the benefit of U.S. provisional patent application serial No. 60/334,760, filed on Nov. 15, 2001.

### FIELD OF THE INVENTION

This invention relates to compositions and methods for inducing a reversible immortalized phenotype in normal cultured cells. In particular, the invention relates to inducing this phenotype with a mutant modified form of cyclin dependent protein kinase.

### BACKGROUND OF THE INVENTION

Growth and division of living cells involve a regular series of events and processes that comprise the cell cycle. The cell cycle is typically described as a set of phases separated by gaps. There are two main phases; the M phase (the time when the cell is dividing) and the interphase (the time when the cell is not dividing). The interphase is subdivided into the time when DNA synthesis is proceeding, known as the S- or synthesis phase, and the gaps that separate the S-phase from mitosis.

The M phase consists of nuclear division (mitosis) and cytoplasmic division (cytokinesis). The process of cytokinesis terminates the M phase and marks the beginning of the interphase of the next cell cycle. The daughter cells resulting from completion of the M phase thus begin the interphase of a new cell cycle.

G1 is the gap after mitosis, but before DNA synthesis starts. G2 is the gap after DNA synthesis is complete, before mitosis begins. Interphase is thus composed of G1, S-phase and G2, and normally comprises 90% or more of the total cell cycle time.

Appropriate growth and differentiation of a cell depends on an orderly progression through the cell cycle. This progression is controlled by a series of positive and negative regulators, such as the cyclins and the cyclin dependent kinases, which act at defined points in the cycle. Processes such as regulated proliferation, differentiation, senescence and apoptosis depend on the proper functioning and interaction of these cell cycle regulators.

For example, the point in G1 at which cells irrevocably commit to DNA synthesis (and thus enter the cell cycle) is controlled by protein complexes consisting of cyclin dependent kinases cdk4 or cdk6, and the D-type cyclins D1, D2 and D3. Association of the D-type cyclins with cdk4 or cdk6 results in catalytic activation of these cyclin dependent kinases. Activated cdk4 or cdk6 in turn phosphorylates the retinoblastoma (Rb) family of proteins.

The Rb proteins negatively regulate the passage of cells from G1 to S-phase by sequestering transcription factors such as E2F, which are critical to the G1/S transition. Phosphorylation of the Rb proteins by activated cdk4 or cdk6 inactivates the Rb proteins, and prevents the sequestration of the transcription factors. The cell then progresses through the G1 block into S-phase.

The cdk4 cyclin dependent kinase is regulated by members of the INK4 family of proteins, in particular the p16^{Ink4a} kinase inhibitor. p16^{Ink4a} appears to associate with cdk4 and prevents the phosphorylation of Rb proteins. Mutations in the p16^{Ink4a} protein abolish the ability of p16^{Ink4a} to associate with cdk4 and block Rb phosphorylation. Such mutations have been implicated in familial melanomas. Ranade et al. (1995), Nat. Gen. 10: 114-116. Deletions and rearrangements in human chromosome 9p21 encompassing the p16^{Ink4a} gene have been associated with various forms of cancer. Noburi et al. (1994), Nature 368: 753-756; Kamb et al. (1994), Science 264: 436-440.

A mutation in the p16^{Ink4a} binding site on cdk4 has also been identified in patients with familial melanoma. The missense mutation results in an exchange of arginine for cysteine at position 24 of the cdk4 protein, with a consequent loss of affinity for p16^{Ink4a}. The mutated cdk4, called cdk4^{R24C}, is still able to bind cyclin D1 and form a functional kinase. See Wolfel et al. (1995), Science 269: 1281-1284 and Zuo et al. (1996), Nat. Gen. 12: 97-99.

Transgenic mice homozygous for the cdk4^{R24C} mutation exhibit an increase in weight of 5-10% as compared to control littermates, an increased population of testicular Leydig cells, and pancreatic islet hyperplasia. However, these mice do not develop melanoma similar to that observed in humans carrying the same cdk4^{R24C} mutation. Rane et al. (1999), Nat. Gen. 22: 44-52.

cdk6 is also inhibited by members of the p16 protein family. A mutation in the p16 protein binding site of cdk6 which converts Arg31 to Cys has been reported in a neuroblastoma cell line; see Easton J et al. (1998), Cancer Res. 58: 2624-2632. This exchange of Arg for Cys reduces binding of p16 inhibitor protein to cdk6.

Progression through the G1 block into the S-phase of the cell cycle is also affected by the cyclin dependent protein kinase cdk2. cdk2 is active when phosphorylated on threonine 160 upon association with either cyclin E (which is expressed during late G1) or cyclin A (which is expressed during S-, G2- and M-phases). Cyclin A or E/cdk2 complexes can be inactivated by phosphorylation on Thr14 or Tyr15 or when associated with inhibitory proteins such as p27KIP1.

It is well known that cells grown in culture experience cell cycle arrest, for example through contact inhibition when the cells become confluent in the culture vessel. Growth-arrested cells can by stimulated to divide by removing them from the culture vessel and seeding them at a lower density into new culture vessels. This process is known as “passaging” the cells. After a given number of passages, certain cell types can undergo replicative senescence and cease growing altogether, regardless of the cell density in the culture vessel. Replicative senescence is a significant problem where large numbers of cells or numerous passages are desired. Still other cell types are difficult to grow in culture at all, regardless of the passage number or cell density.

Therefore, it is sometimes desirable to deliberately perturb the cell cycle of cultured cells, so that the cells do not undergo cell cycle arrest, replicative senescence or apoptosis. Ideally, the cells would exhibit an immortalized phenotype; that is, would maintain constant proliferation rates without displaying any morphological features of senescing cells. The deliberately “immortalized” cells would continuously progress through successive cell cycles, and generate large numbers of cells and high passage numbers. For example, one may wish to induce the immortalized phenotype in order to grow large numbers of cells for experimentation or transplantation. Likewise, one may wish to grow large numbers of cells that produce, either naturally or by design, a compound of therapeutic or commercial interest.

However, the continuously growing cells should not exhibit a transformed (i.e., cancerous) phenotype, especially if the cells are to be used therapeutically. The reversibly “immortalized,” but non-transformed, cells can also be used as a background for testing possible carcinogens. It is therefore desirable to induce a state of continuous growth in the cultured cells that is not permanent. It is also desirable that the reversible state of continuous growth is inducible without extensive manipulation of the cultured cells. Techniques which require extensive manipulation (e.g., transfection by nucleic acids) are expensive, increase the risk of culture contamination, and are often ineffective. Moreover, the expression of exogenous genetic material in cultured cells is notoriously difficult to control.

Continuously growing cells, especially cells of higher mammals, can experience shortening of chromosome telomeres with each cell cycle. Eventually, this shortening reaches critical levels and causes the cell to undergo replicative senescence and cell death by apoptosis. Cell death can occur even though the cells have been induced to progress continuously through the cell cycle. Thus, to proliferate beyond this senescent checkpoint, telomere length must be restored and maintained above a threshold level. In some embryonic stem cells and tumor cells, telomere length is preserved by an enzyme called telomerase. There is some evidence that expressing the gene for TERT, which encodes the catalytic subunit of telomerase, can induce telomere lengthening in telomerase-negative cells. See Hodes R J (1999), J. Exp. Med. 190: 153-156 and Urquidi et al. (2000), An. Rev. Med. 51: 65-79.

There is thus a need for compositions and methods which induce a state of continuous growth in cultured cells without extensive manipulation of cells, wherein the state of continuous growth is not a permanent characteristic of the cells but may be reversed easily and at will.

### SUMMARY OF THE INVENTION

It has now been found that cdk4 or cdk6 proteins, or biologically active fragments, derivatives, homologs or analogs thereof, that contain mutations which prevent binding of inhibitor proteins can induce a reversible state of continual growth in cultured cells when administered exogenously to the cells in culture.

It has also been found that cdk2 proteins, or biologically active fragments, derivatives, homologs or analogs thereof, that contain mutations which prevent cdk2 inactivation while allowing binding with cyclins A or E can induce a reversible state of continual growth in cultured cells when administered exogenously to the cells in culture.

Thus, the invention provides a continual growth-inducing composition comprising at least one compound comprising a cdk2, cdk4 or cdk6 protein, or biologically active fragment, derivative, homolog or analog thereof, having an activating mutation and one or more modifications which allow the compound to enter a cell when administered exogenously to a cell in culture. The one or more modifications can comprise a leader sequence which directs entry of the compound into the cell. The composition can additionally comprise a compound comprising the catalytic subunit of telomerase, or a biologically active fragment, derivative, homolog or analog thereof, which also has one or more modifications which allow it to enter a cell when administered exogenously to a cell in culture. Preferably, the leader sequence is designed so that it is cleaved from the compound inside the cell.

The invention also provides a method of inducing a reversible state of continuous growth in cultured cells, comprising the steps of:

providing a culture of viable cells;

contacting the cells with an effective amount of a composition comprising at least one compound comprising a cdk2, cdk4 or cdk6 protein, or biologically active fragment, derivative, homolog or analog thereof, having an activating mutation and one or more modifications which allow the compound to enter the cells, so that a state of continuous growth is induced for as long as the cells are in contact with the composition; and

optionally reversing the state of continuous growth by removing the compound from contact with the cells.

The invention provides a method of screening an agent for the ability to transform cultured cells, comprising the steps of:

providing a culture of viable cells;

contacting the cells with an effective amount of a composition comprising at least one compound comprising a cdk2, cdk4 or cdk6 protein, or biologically active fragment, derivative, homolog or analog thereof, having an activating mutation and one or more modifications which allow the compound to enter the cells, so that a state of continuous growth is induced for as long as the cells are in contact with the composition;

contacting the cells with an agent; and

evaluating the cells for the presence of a transformed phenotype.

In one embodiment, the agent to be evaluated comprises a chemical carcinogen, mutagen or teratogen. In another embodiment, the agent to be evaluated comprises nucleic acid sequences encoding, for example, a potential oncogene.

The invention also provides a cultured cell in which a reversible state of continual growth has been induced by a composition comprising at least one compound comprising a cdk2, cdk4 or cdk6 protein, or biologically active fragment, derivative, homolog or analog thereof having an activating mutation and one or more modifications which allow the compound to enter the cell when administered exogenously.

### Amino Acid Abbreviations

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified. In the amino acid structure formulae, each residue is generally represented by a one-letter or three-letter designation, corresponding to the trivial name of the amino acid, in accordance with the following schedule:

| | | |
|---|---|---|
| A | Alanine | Ala |
| C | Cysteine | Cys |
| D | Aspartic Acid | Asp |
| E | Glutamic Acid | Glu |
| F | Phenylalanine | Phe |
| G | Glycine | Gly |
| H | Histidine | His |
| I | Isoleucine | Ile |
| K | Lysine | Lys |
| L | Leucine | Leu |
| M | Methionine | Met |
| N | Asparagine | Asn |
| P | Proline | Pro |
| Q | Glutamine | Gln |
| R | Arginine | Arg |
| S | Serine | Ser |
| T | Threonine | Thr |
| V | Valine | Val |
| W | Tryptophan | Trp |
| Y | Tyrosine | Tyr |

### Definitions

The following definitions, of terms used throughout the specification, are intended as an aid to understanding the scope and practice of the present invention.

By “cdk4 activating mutation” or “cdk6 activating mutation” is meant a mutation in a cdk4 or cdk6 protein or biologically active fragment, homolog, derivative or analog thereof, which prevents binding of an inhibitor (e.g., a p16 protein, such as p16^{Ink4a} or an equivalent inhibitor) but does not affect cdk4 or cdk6 kinase activity.

By “cdk2 activating mutation” is meant a mutation in a cdk2 protein or biologically active fragment, homolog, derivative or analog thereof, which prevents phosphorylation of cdk2 at Thr14 or Tyr15, and/or prevents binding of cdk2 to inhibitory proteins such as p27KIP1, but which does not affect cdk2 kinase activity.

“Biologically active”, when referring to a cdk2, cdk4 or cdk6 protein, or a fragment, derivative, homolog or analog thereof, means the ability to induce continual growth as measured by the cell culture assay of Example 4 below.

“Biologically active”, when referring to a TERT protein, or a fragment, derivative, homolog or analog thereof, means the ability to extend or maintain telomeric sequences as measured by the telomeric repeat amplification protocol (TRAP) assay described in Example 9 below.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half life without adversely affecting their activity. Additionally, a disulfide linkage can be present or absent in the peptides of the invention.

Amino acids have the following general structure:

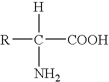

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

“Amino-terminal truncation fragment” with respect to an amino acid sequence means a fragment obtained from a parent sequence by removing one or more amino acids from the amino-terminus thereof.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

“Carboxy-terminal truncation fragment” with respect to an amino acid sequence means a fragment obtained from a parent sequence by removing one or more amino acids from the carboxy-terminus thereof.

“Derivative” includes any purposefully generated peptide which in its entirety, or in part, has a substantially similar amino acid sequence to a protein. Derivatives can be characterized by single or multiple amino acid substitutions, deletions, additions, or replacements. These derivatives can include (a) derivatives in which one or more amino acid residues of a protein are substituted with conservative or non-conservative amino acids, (b) derivatives in which one or more amino acids are added to a protein, (c) derivatives in which one or more of the amino acids of a protein includes a substituent group, (d) derivatives in which a protein or a portion thereof is fused to another peptide (e.g., serum albumin), (e) derivatives in which one or more nonstandard amino acid residues (i.e., those other than the 20 standard L-amino acids commonly found in naturally occurring proteins) are incorporated or substituted into the a protein sequence, and (f) derivatives in which one or more nonamino acid linking groups are incorporated into or replace a portion of a protein.

“Fragment” refers to a portion of the a protein amino acid sequence comprising at least two amino acid residues, and which retains biological activity. Fragments can be generated by amino-terminal truncation, carboxy-terminal truncation or both of these. Fragments can also be generated by chemical or enzymatic digestion or peptide synthesis.

“Homolog” includes any nonpurposely generated peptide which in its entirety, or in part, has a substantially similar amino acid sequence to a protein and has an activating mutation. Homologs can include paralogs, orthologs, and naturally occurring alleles or variants of a protein.

By “libraries” is meant pools and subpools of pro-analogs.

“Peptide” and “protein” are used interchangeably, and refer to a compound comprised of amino acid residues covalently linked by peptide bonds. A protein or peptide must contain at least two amino acids and no limitation is placed on the maximum number of amino acids which can comprise a protein's or peptide's sequence. The amino acids of the peptides described herein and in the appended claims are understood to be either D or L amino acids with L amino acids being preferred.

“Variant” as the term is used herein, is a nucleic acid or peptide that differs from a reference nucleic acid or peptide respectively, but retains essential properties. Changes in the sequence of a nucleic acid variant might not alter the amino acid sequence of a peptide encoded by the reference nucleic acid, or can result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or it can be a variant that is not known to occur naturally. Non-naturally occurring variants of nucleic acids and peptides can be made by mutagenesis techniques or by direct synthesis.

As used herein, a peptide or a portion of a peptide which has a “substantially similar amino acid sequence” to a reference protein means the peptide, or a portion thereof, has an amino acid sequence identity or similarity to the reference protein of greater than about 80%. Preferably, the sequence identity is greater than about 85%, more preferably greater than about 90%, particularly preferably greater than about 95%, and most preferably greater than about 98%. Amino acid sequence similarity or identity can be computed by using the BLASTP and TBLASTN programs which employ the BLAST (basic local alignment search tool) 2.0.14 algorithm; BLASTP and TBLASTN settings to be used in such computations are indicated in Table 1 below. Amino acid sequence identity is reported under “Identities” by the BLASTP and TBLASTN programs. Amino acid sequence similarity is reported under “Positives” by the BLASTP and TBLASTN programs. Techniques for computing amino acid sequence similarity or identity are well known to those skilled in the art, and the use of the BLAST algorithm is described in Altschul et al. (1990), J. Mol. Biol. 215: 403-10 and Altschul et al. (1997), Nucleic Acids Res. 25:3389-3402, the disclosures of which are herein incorporated by reference in their entirety. BLASTP and TBLASTN programs utilizing the BLAST 2.0.14 algorithm and can be accessed through the National Center for Biotechnology Information website maintained by the National Institutes of Health and the National Library of Medicine.

TABLE 1
Settings to be used for the computation of amino acid sequence
similarity or identity with BLASTP and TBLASTN programs utilizing the
BLAST 2.0.14 algorithm.

| | |
|---|---|
| Expect Value | 10 |
| Filter | Low complexity filtering using SEG program* |
| Substitution Matrix | BLOSUM62 |
| Gap existence cost | 11 |
| Per residue gap cost | 1 |
| Lambda ratio | 0.85 |
| Word size | 3 |

*The SEG program is described by Wootton and Federhen (1993), Comput. Chem. 17:149-163.

“Substantially similar nucleic acid sequence” means a nucleic acid sequence corresponding to a reference nucleic acid sequence wherein the corresponding sequence encodes a peptide having substantially the same structure and function as the polypeptide encoded by the reference nucleic acid sequence; e.g., where only changes in amino acids not affecting the polypeptide function occur. Preferably, the substantially similar nucleic acid sequence encodes the peptide encoded by the reference nucleic acid sequence. The percentage of identity between the substantially similar nucleic acid sequence and the reference nucleic acid sequence is, for example, at least 80%. Preferably, the sequence identity is at least about 85%, more preferably at least about 90%, particularly preferably at least about 95%, most preferably at least about 99%. Substantial similarity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleic acid sequence is substantially similar to a reference nucleic acid sequence are: 1) for low stringency—50% formamide; 5×Standard Saline Citrate (SSC) (20×SSC=3M NaCl; 0.3 M sodium citrate 2H₂O; pH 7.0); 20 mM sodium phosphate; 5× Denhardt's solution (100× Denhardt's solution=10 g Ficoll 400; 10 g polyvinylpyrollidone; 10 g bovine serum albumin fraction V; H₂O to 100 ml); 10% sodium dextran sulfate; 100 micrograms/ml denatured salmon sperm DNA; rotating at 37° C. overnight in a hybridization oven, with washing 3× for 20 min. in 2×SSC; 0.1% sodium dodecyl sulfate (SDS) at 37° C., followed by washing 3× in 0.1% SSC; 0.1% SDS at 37° C.; 2) for medium stringency—50% formamide; 5×SSC; 20 mM sodium phosphate; 5× Denhardt's solution; 10% sodium dextran sulfate; 100 micrograms/ml denatured salmon sperm DNA; rotating at 42° C. overnight in a hybridization oven, with washing 3× for 20 min. in 2×SSC; 0.1% sodium dodecyl sulfate (SDS) at 42° C., followed by washing 3× in 0.1% SSC; 0.1% SDS at 42° C.; 3) for high stringency—50% formamide; 5×SSC; 20 mM sodium phosphate; 5× Denhardt's solution; 10% sodium dextran sulfate; 100 micrograms/ml denatured salmon sperm DNA; rotating at 55° C. overnight in a hybridization oven, with washing 3× for 20 min. in 2×SSC; 0.1% sodium dodecyl sulfate (SDS) at 55° C., followed by washing 3× in 0.1% SSC; 0.1% SDS at 55° C. Suitable computer algorithms to determine substantial similarity between two nucleic acid sequences include, but are not limited to: GCS program package (Devereux et al. (1984), Nucl. Acids Res. 12: 387), and the BLASTN, FASTA programs (Altschul et al. (1990), J. Molec. Biol. 215: 403) or “BLAST 2 Sequences” alignment program available through the National Center for Biotechnology Information website maintained by the National Institutes of Health and the National Library of Medicine.

The default settings provided with these programs are adequate for determining substantial similarity of nucleic acid sequences for purposes of the present invention.

“Substantially purified” refers to a population of peptides or cells which is substantially homogenous in character due to the removal of other compounds (e.g., other peptides, nucleic acids, carbohydrates, lipids) or other cells originally present. “Substantially purified” is not meant to exclude artificial or synthetic mixtures with other compounds, or the presence of impurities which do not interfere with biological activity, and which might be present, for example, due to incomplete purification, addition of stabilizers, or formulation into a pharmaceutically acceptable preparation.

“Synthetic mutant” includes any purposefully generated mutant or variant derived from a protein. Such mutants can be purposefully generated by, for example, chemical mutagenesis, polymerase chain reaction (PCR) based approaches, or primer based mutagenesis strategies well known to those skilled in the art.

“Transfection” is the introduction of a nucleic acid into a cell.

“Transformed” or “transformation” refers to the induction of a phenotype in cultured cells characterized at least by non-reversible, uncontrolled growth, the ability to form foci, and attachment-independent growth.

A “nucleic acid sequence” is a linear segment of single- or double-stranded DNA or RNA that can be isolated from any source. In the context of the present invention, the nucleic acid molecule is preferably a segment of DNA.

A “gene sequence” is a nucleic acid sequence capable of directing expression of a particular nucleic acid sequence in an appropriate host cell, comprising a promoter operably linked to the nucleic acid sequence of interest which is operably linked to termination signals. A gene sequence also typically comprises nucleic acid sequences required for proper translation of the expressed nucleic acid sequence. The gene sequence can be chimeric, meaning that at least one of its components is heterologous with respect to at least one of its other components. The gene sequence can also be one which is naturally occurring but has been obtained in a recombinant form useful for expression in cultured cells. Typically, however, the gene sequence is heterologous with respect to the host, i.e., the particular nucleic acid sequence of the expression cassette does not occur naturally in the host cell and must have been introduced into the host cell or an ancestor of the host cell by a transfection event. The expression of the gene sequence can be under the control of a constitutive promoter, or of an inducible promoter which initiates transcription only when the host cell is exposed to some particular external stimulus.

“Operatively linked” refer to two nucleic acid sequences that are related physically or functionally. For example, a promoter or regulatory DNA sequence is said to be “operatively linked” to DNA sequence that produces an RNA or encodes a protein if the two sequences are operatively linked, or situated such that the regulator DNA sequence will affect the expression level of the coding or structural DNA sequence.

A “promoter” is an untranslated nucleic acid sequence upstream of the coding region that contains the binding site for RNA polymerase and initiates transcription of RNA. The promoter region can also include other regulatory elements.

“Regulatory elements” refer to sequences involved in controlling the expression of a gene sequence. Regulatory elements comprise a promoter operably linked to the nucleic acid sequence of interest and termination signals. They also typically encompass sequences required for proper translation of a gene sequence.

### BRIEF DESCRIPTION OF THE FIGURES

FIGS. 1A and 1B—Cell Growth Characteristics of cdk4^{R24C/R24C} Mouse Embryonic Fibroblasts (MEFs). FIG. 1A: 3×10⁵ MEF cells (passage<4) from cdk4^{+/+} (open circles), cdk4^{+/R24C} (open squares) and cdk4^{R24C/R24C} (closed circles) mice were grown for the indicated time in days, and the number of viable cells were counted using trypan blue exclusion analysis. FIG. 1B: 3×10⁵ MEF cells (passage<4) from cdk4^{+/+} (+/+, open bars), cdk4^{+/R24C} (+/R24C, hatched bars) and cdk4^{R24C/R24C} (R24C/R24C, solid bars) mice were exponentially cultured for 24 hours and the percentage of cells in G0/G1, S and G2/M were determined by FACs analysis upon staining with propidium iodide.

FIG. 2—Escape From Senescence and Immortalization in cdk4^{R24C/R24C} MEFs. cdk4^{+/+} (open circles; n=4), cdk4^{+/R24C} (open squares; n=4) and cdk4^{R24C/R24C} (closed circles; n=4) MEF cultures were propagated in DMEM media supplemented with 10% FBS for the indicated passages according to the 3T3 protocol. The graph shows the accumulated number of doublings that representative cultures have undergone during 20 successive passages.

### DETAILED DESCRIPTION OF THE INVENTION

The present invention provides a composition which, when added exogenously to cultured cells, reversibly induces continual cell growth. The continual cell growth is induced for as long as the composition is in contact with the cultured cells.

The continual growth-inducing composition of the invention comprises at least one compound comprising a cdk2, cdk4 or cdk6 cyclin dependent kinase protein, or a biologically active fragment, derivative, homolog or analog thereof, that has an activating mutation. The compounds of the invention are therefore constitutively active kinases inside a cultured cell. The compounds can further comprise modifications which allow rapid and efficient transport into the cultured cell by simply contacting the compounds with the cells, so that extensive manipulations of the cultured cells are not required.

As used herein, “cdk4 protein” or “cdk6 protein” includes the cdk4 or cdk6 protein from any species. cdk4 and cdk6 cDNA sequences from various species are listed in Table 2. One of ordinary skill can readily identify nucleic acid sequences from other species as encoding cdk4 or cdk6 proteins, based on similarity to the sequences listed in Table 2. Nucleic acid sequences that exhibit substantial similarity to the Table 2 sequences can be considered as encoding cdk4 or cdk6 proteins, and can be used to derive compounds comprising a continual growth-inducing composition according to the invention, as described in detail below. Extant proteins can also be identified as cdk4 or cdk6 proteins by comparison to the protein sequences listed in Table 2.

TABLE 2
cdk4 and cdk6 cDNA and Protein Sequences

| Encoded | | GenBank Acc. | | |
|---|---|---|---|---|
| Protein | Species | No.¹ | SEQ ID NO: | |
| cdk4(wt²) | Homo sapiens³ | XM053138 | protein | 1 |
| | | | cDNA | 2 |
| cdk4(wt) | Homo sapiens³ | XM048541 | protein | 3 |
| | | | cDNA | 4 |
| cdk4(wt) | Homo sapiens³ | XM048543 | protein | 5 |
| | | | cDNA | 6 |
| cdk4(wt) | Mus musculus | NM009870 | protein | 7 |
| | (Balb/C) | | cDNA | 8 |
| cdk4(wt) | Rattus norvegicus | L11007 | protein | 9 |
| | (rat) | | cDNA | 10 |
| cdk4(wt) | Sus scrofa | U68478 | protein | 11 |
| | (domestic pig) | | cDNA | 12 |
| cdk4(wt) | Xenopus laevis | X89477 | protein | 13 |
| | (African clawed frog) | | cDNA | 14 |
| cdk4^{R24C} | Homo sapiens | Z48970 | protein | 15 |
| (mutant) | | | cDNA | 16 |
| cdk6 | Homo sapiens | XM_004987 | protein | 17 |
| | | | cDNA | 18 |
| cdk6 | Homo sapiens | | protein | 19 |
| | (mutant) | | cDNA | 20 |
| cdk6 | Mus musculus | AF132483 | protein | 21 |
| | (Balb/C) | | cDNA | 22 |
| cdk6 | Rattus norvegicus | AF352168 | protein | 23 |
| | (rat) | | cDNA | 24 |
| cdk6 | Gallus gallus | L77991 | protein | 25 |
| | (chicken) | cDNA | 26 | |

¹GenBank records identified in Table 2 are herein incorporated by reference.
²wt-wild type
³The three listed H. sapiens cdk4 protein are allelic variants.

The primary amino acid sequences of the allelic variants of normal human cdk4 protein are given in SEQ ID NOS: 1, 3 and 5. Normal human cdk4 is 303 amino acids long, and comprises a p16^{Ink4a} binding site which includes the Arg residue at position 24 (Arg2⁴).

A preferred activating mutation is the exchange of the conserved Arg residue for Cys in the p16^{Ink4a} binding site of cdk4. This activating mutation, called “cdk4^{R24C}” in humans, is described by Wolfel et al. (1995), Science 269: 1281-1284 and Zuo et al. (1996), Nat. Gen. 12: 97-99, the entire disclosures of which are herein incorporated by reference. The primary amino acid sequence of cdk4^{R24C} is given in SEQ ID NO: 15.

Other mutants of the cdk4 protein which have similar biological activity to cdk4^{R24C} (i.e., cdk4 activating mutations) can be generated by random or site-directed mutagenesis, protein engineering, recombinant DNA technology, or a combination of these techniques.

One or more specific cdk4 amino acids for mutation may be chosen, based on the known sequence, structure and function of the cdk4 or cdk4^{R24C} protein. For example, inspection of the cdk4 amino acid sequences encoded by the cDNAs listed in Table 2 shows that this protein is highly conserved among diverse species. Because of this conservation, one skilled in the art can readily identify amino acids in any cdk4 protein which, when mutated, would be expected to produce activating mutations.

For example, each listed cdk4 protein (with the exception of mutant cdk4^{R24C}) has a conserved Arg residue at the p16^{Ink4a} binding site (position 24 in the human, mouse, rat and pig; position 27 in X. laevis). Thus, exchanging the conserved Arg at the p16^{Ink4a} binding site for a Cys or another amino acid residue, analogous to the human cdk4^{R24C} mutation, would result in an activating mutation in the cdk4 protein from any species.

Comparison of, for example, the X. laevis and human cdk4 sequences shows that a number of amino acid residues around the conserved Arg are also highly conserved. Sequence comparisons suitable for identifying conserved cdk4 sequences can be performed, for example, with publicly available alignment algorithms such as the “BLAST 2 Sequences” alignment program described above.

A conserved amino acid sequence around the conserved Arg residue at the cdk4 protein p16^{Ink4a} binding site, as identified by BLAST 2 Sequence alignment, is:

(SEQ ID NO: 27)

Tyr-Glu-Pro-Val-Ala-Glu-Ile-Gly-Val-Gly-Ala-Tyr-
Gly-Thr-Val-Tyr-Lys-Ala-Arg-Asp-Xaa₁-Xaa₂-Ser-Gly-
Xaa₃-Phe-Val-Ala-Leu-Lys-Xaa₄-Val-Arg-Val.

The conserved Arg residue is underlined, and Xaa₁, Xaa₂, Xaa₃ and Xaa₄ represent any amino acid. Preferably, Xaa₁ is Leu or Pro; Xaa₂ is Glu or His; Xaa₃ is Lys or His; and/or Xaa₄ is Asn or Ser. It can be seen in the forgoing preferred amino acid pairs that the choice between Asn or Ser for Xaa₄ represents a conservative substitution. Without wishing to be bound by any theory, the sequence conservation around the conserved Arg residue in SEQ ID NO: 27 indicates the importance of the conserved residues in p16^{Ink4a} binding. One of ordinary skill in the art can readily choose one or more of the amino acids in SEQ ID NO: 27 for mutation, in order to produce a mutation in any cdk4 protein that prevents binding of inhibitor protein but allows kinase activity (i.e., an activating mutation). The cell culture assay described in Example 4 below can be used to confirm that a given mutation in this conserved sequence results in a cdk4 activating mutation.

The primary amino acid sequence of the normal human cdk6 protein is given in SEQ ID NO: 17. Normal human cdk6 is 326 amino acids long, and comprises a conserved Arg residue in a p16 inhibitory protein binding site, analogous to cdk4 Arg24, at position 31 (Arg31). A preferred cdk6 activating mutation is the exchange of the conserved Arg residue for Cys in cdk6, as described in Easton J et al. (1998), Cancer Res. 58: 2624-2632, the disclosure of which is herein incorporated by reference.

Comparison of the human and chicken cdk6 sequences in Table 2 shows that all of the amino acid residues around the conserved Arg in the p16 protein binding site are conserved. Sequence comparisons suitable for identifying conserved cdk6 sequences can be performed, for example, with publicly available alignment algorithms such as the “BLAST 2 Sequences” alignment program described above.

A conserved amino acid sequence around the conserved Arg residue at the cdk6 proteins, as identified by BLAST 2 Sequence alignment, is:

(SEQ ID NO: 28)

Tyr Glu Cys Val Ala Glu Ile Gly Glu Gly Ala Tyr
Gly Lys Val Phe Lys Ala Arg Asp Leu Lys Asn Gly
Gly Arg Phe Val Ala Leu Lys Arg Val Arg Val.

The conserved Arg residue is underlined. Without wishing to be bound by any theory, the sequence conservation around the conserved Arg residue in SEQ ID NO: 28 indicates the importance of the conserved residues in p16 inhibitor protein binding. One of ordinary skill in the art can readily choose one or more of these amino acids for mutation in order to produce a mutation in any cdk6 protein that prevents inhibitor binding but allows kinase activity (i.e., an activating mutation). The cell culture assay described in Example 4 below can be used to confirm that a mutation in this conserved sequence results in a cdk6 activating mutation.

Amino acids in cdk4 or cdk6 proteins which can be mutated to produce activating mutations can also be identified by comparison of the human cdk4 and cdk6 sequences around their respective conserved Arg residues. Sequence comparisons suitable for identifying conserved cdk6 sequences can be performed, for example, with publicly available alignment algorithms such as the “BLAST 2 Sequences” alignment program described above.

A conserved amino acid sequence around the conserved Arg residues in the cdk4 and cdk6 proteins, as identified by BLAST 2 Sequence alignment, is:

(SEQ ID NO: 29)

Tyr Glu Xaa₁ Val Ala Glu Ile Gly Xaa₂ Gly Ala Tyr
Gly Xaa₃ Val Xaa₄ Lys Ala Arg Asp Xaa₅ Xaa₆ Xaa₇
Gly Xaa₈ Phe Val Ala Leu Lys Xaa₉ Val Arg Val.

The conserved Arg residue is underlined, and Xaa₁ through Xaa₉ represent any amino acid. Preferably, Xaa₁ is Cys or Pro; Xaa₂ is Glu or Val; Xaa₃ is Lys or Thr; Xaa₄ is Phe or Tyr; Xaa₅ is Lys or Pro; Xaa₆ is Asn or His; Xaa₇ is Gly or Ser; Xaa₈ is Arg or His; and/or Xaa₉ is Arg or Ser. It can be seen in the forgoing preferred amino acid pairs that the choices for Xaa₄, Xaa₅, Xaa₇, Xaa₈, and Xaa₁₀ represent conservative substitutions. Without wishing to be bound by any theory, the sequence conservation around the conserved Arg residue in SEQ ID NO: 29 indicates the importance of the conserved residues in constructing activating mutations. One of ordinary skill in the art can readily choose one or more of these amino acids for mutation in order to produce a mutation that prevents inhibitor binding but allows kinase activity in any cdk4 or cdk6 protein (i.e., an activating mutation). The cell culture assay described in Example 4 below can be used to confirm that a mutation in this conserved sequence results in a cdk4 or cdk6 activating mutation.

As used herein, “cdk2 protein” includes the cdk2 protein from any species. cdk2 cDNA and protein sequences from various species are listed in Table 3. One of ordinary skill can readily identify nucleic acid sequences from other species as encoding cdk2 proteins, based on similarity to the sequences listed in Table 3. Nucleic acid sequences that exhibit substantial similarity to the Table 3 sequences can be considered as encoding cdk2 proteins, and can be used to derive compounds used in a continual growth-inducing composition according to the invention, as described in detail below. Extant proteins can also be identified as cdk2 proteins by comparison to the protein sequences listed in Table 3.

TABLE 3
cdk2 cDNA and Protein Sequences

| Encoded | | GenBank | | |
|---|---|---|---|---|
| Protein | Species | Acc. No.¹ | SEQ ID NO: | |
| cdk2 | Homo sapiens | XM_049152 | Protein | 30 |
| | | | cDNA | 31 |
| cdk2 | Homo sapiens | N/A² | Protein | 32 |
| | (mutant) | | cDNA | 33 |
| cdk2 | Mus musculus | NM_016756 | Protein | 34 |
| | (mouse) | | cDNA | 35 |
| cdk2L | Mus musculus | AJ223732 | Protein | 36 |
| | (mouse) | | cDNA | 37 |
| cdk2 | Mesocricetus auratus | D17350 | Protein | 38 |
| | (golden hamster) | | cDNA | 39 |
| cdk2L | Mesocricetus auratus | D17351 | Protein | 40 |
| | (golden hamster) | | cDNA | 41 |
| cdk2 | Cricetulus griseus | AJ223949 | Protein | 42 |
| | (Chinese hamster) | | cDNA | 43 |
| cdk2L | Cricetulus griseus | AJ223952 | Protein | 44 |
| | (Chinese hamster) | | cDNA | 45 |
| cdk2-alpha | Rattus norvegicus | D28753 | Protein | 46 |
| | (rat) | | cDNA | 47 |
| cdk2-beta | Rattus norvegicus | D63162 | Protein | 48 |
| | (rat) | | cDNA | 49 |
| cdk2 | Carassius auratus | S40289 | Protein | 50 |
| | (goldfish) | | cDNA | 51 |
| cdk2 | Sphaerechinus granularis | AJ224917 | Protein | 52 |
| | (sea urchin) | | cDNA | 53 |
| cdk2 | Xenopus laevis | U07979 | Protein | 54 |
| | (African clawed frog) | | | |

¹GenBank records identified in Table 3 are herein incorporated by reference.
²Not applicable

The primary amino acid sequence of the normal human cdk2 protein is given in SEQ ID NO: 30. Normal human cdk2 is 298 amino acids long, and comprises a conserved Arg residue at position 22 (Arg22) analogous to cdk4 Arg24 and cdk6 Arg31. A suitable cdk2 activating mutation is the exchange of the conserved Arg residue for Cys in humans. In mice, rats, hamster X. laevis (African clawed frog) and Carassius auratus (goldfish), the conserved Arg is replaced by a Lysine at position 22. In these species, a longer “beta” form of cdk2, comprising 346 amino acids, has been observed (see Table 3). The beta form of cdk2 still has the conserved Lys at position 22. Thus, another activating mutation is the exchange of Lys22 for Cys in the alpha or beta form of cdk2 from these species.

Other activating mutations comprise alterations in the known binding site for inhibitory proteins such as p27KIP1, or alterations in the amino acid residues which are phosphorylated to inactivate cdk2; i.e., Thr 14 and Tyr15.

Comparison of the human cdk2 and human cdk4 sequences shows that a number of amino acid residues around the conserved Arg are also highly conserved. Sequence comparisons suitable for identifying conserved cdk2 and cdk4 sequences can be performed, for example, with publicly available alignment algorithms such as the “BLAST 2 Sequences” alignment program described above.

A conserved amino acid sequence around the conserved Arg residue at the human cdk2 and cdk4 proteins, as identified by BLAST 2 Sequence alignment, is:

(SEQ ID NO: 55)

Xaa₁ Xaa₂ Xaa₃ Val Xaa₄ Xaa₅ Ile Gly Xaa₆ Gly Xaa₇
Tyr Gly Xaa₈ Val Tyr Lys Ala Arg Xaa₉ Xaa₁₀ Xaa₁₁
Xaa₁₂ Gly Xaa₁₃ Xaa₁₄ Val Ala Leu Lys Xaa₁₅ Xaa₁₆
Arg.

The conserved Arg residue is underlined, and Xaa₁ through Xaa₁₆ represent any amino acid. Preferably, Xaa₁ is Phe or Pro; Xaa₂ is Glu or Gln; Xaa₃ is Lys or Pro; Xaa₄ is Glu or Ala; Xaa₅ is Lys or Glu; Xaa₆ is Glu or Val; Xaa₇ is Thr or Ala; Xaa₈ is Val or Thr; Xaa₉ is Asn or Asp; Xaa₁₀ is Lys or Pro; Xaa₁₁ is Leu or His; Xaa₁₂ is Thr or Ser; Xaa₁₃ is Glu or His; Xaa₁₄ is Val or Phe; Xaa₁₅ is Lys or Ser; and/or Xaa₁₆ is Ile or Arg. It can be seen in the forgoing preferred amino acid pairs that the choices for Xaa₁, Xaa₂, Xaa₉, Xaa₁₂, Xaa₁₃, and Xaa₁₆ represent conservative substitutions. Without wishing to be bound by any theory, the sequence conservation around the conserved Arg residue in SEQ ID NO: 55 indicates the importance of the conserved residues in generating cdk2 activating mutations. One of ordinary skill in the art can readily choose one or more of these amino acids for mutation in order to produce a mutation that prevents inhibitor binding but allows kinase activity in any cdk2 protein (i.e., an activating mutation). The cell culture assay described in Example 4 below can be used to confirm that a mutation in this conserved sequence results in a cdk2 activating mutation.

Amino acids in cdk2, cdk4 or cdk6 proteins which can be mutated to produce activating mutations can also be identified by other means. For example, the three-dimensional structure of the proteins can be determined by protein X-ray crystallography and/or multidimensional NMR spectroscopy. These spectrographic techniques can be coupled with computer algorithms which can model how changes in the amino acid residues might affect the structure and function of the proteins (see, for example, Nilsson et al. (1992), Curr. Opin. in Struc. Biol. 2: 569-575; Presta (1992), Curr. Opin. in Struc. Biol 2: 593-596; Cech (1992), Curr. Opin. in Struc. Biol. 2: 605-609; and Pickersgill and Goodenough (1991), Trends in Food Sci. and Tech. 5: 122-126). Alternatively, modeling algorithms alone can be used without spectrographic studies to identify amino acid residues important for cdk2, cdk4 or cdk6 protein function. In particular, the regions surrounding the conserved Arg or Lys residue discussed above can be targeted for modification.

Thus, it is understood that the present invention encompasses not only continual growth-inducing compounds comprising the cdk4^{R24C} protein sequence, but also other mutants of cdk2, cdk4 or cdk6 that exhibit biological activity similar to cdk4^{R24C}, particularly with respect to the ability to reversibly induce continual growth in cultured cells.

Having established an amino acid(s) in cdk2, cdk4 or cdk6 to be modified, an altered nucleic acid sequence can be prepared which encodes a protein with the desired amino acid change. The mutant protein itself can also be synthesized directly from amino acid substrates, as described more filly below. However, the present discussion will focus on generating proteins via recombinant nucleic acid technology.

A nucleic acid sequence containing the desired alterations in the cdk2, cdk4 or cdk6 protein can be synthesized de novo, or an unaltered nucleic acid sequence can be synthesized or isolated for subsequent mutagenesis. The nucleic acid sequence can then be subcloned into the appropriate vector for propagation in an appropriate host.

The subcloned nucleic acid sequences can then be expressed directly to generate the altered protein, or (if beginning with the unaltered sequence) subjected to site directed mutagenesis. Suitable nucleic acid coding sequences for producing mutated cdk2, cdk4 or cdk6 proteins with activating mutations are given in Tables 2 and 3 above. One of ordinary skill can readily identify other nucleic acid sequences which encode cdk2, cdk4 or cdk6 proteins based on similarity to the sequences listed in Tables 2 and 3. Nucleic acid sequences that exhibit substantial similarity to the sequences of Tables 2 and 3 can be considered cdk2, cdk4 or cdk6 protein coding sequences, and can be used to derive continual growth-inducing compounds according to the present invention.

Intracellularly produced cdk2, cdk4 or cdk6 protein mutants can be obtained from the host cell by cell lysis, or by using heterologous signal sequences fused to the protein which cause secretion of the protein into the surrounding medium. Preferably, the signal sequence is designed so that it can be removed by chemical or enzymatic cleavage, as described below for the PTD sequences. The proteins thus produced can then be purified by affinity chromatography utilizing tags incorporated into the construct including, but not limited to, 6×His, GST or Myc.

The techniques used to transform cells, construct vectors, construct oligonucleotides, perform site-specific mutagenesis, and the like are widely practiced in the art, and most practitioners are familiar with the standard resource materials which describe specific conditions and procedures. However, the following discussion is presented as a guideline for generating cdk2, cdk4 or cdk6 proteins with activating mutations, according to the present invention. All documents cited in the following discussion are incorporated by reference.

Both prokaryotic and eukaryotic systems can be used to express the nucleic acid sequences encoding the cdk2, cdk4 or cdk6 protein mutants. Prokaryotic hosts are preferred, for example various strains of E. coli. However, other microbial strains can also be used. Plasmid vectors which contain replication sites, selectable markers and regulatory sequences derived from a species compatible with the host are preferred.

Particularly preferred are bacterial plasmid expression systems which utilize regulatory systems compatible with E. coli cells. For example, E. coli can be transformed using derivatives of pBR322, a plasmid derived from an E. coli species by Bolivar et al. (1977), Gene 2: 95. Plasmid pBR322 contains genes for ampicillin and tetracycline resistance, and thus provides multiple selectable markers which can be either retained or destroyed in constructing the desired vector. Other suitable plasmid vectors include plasmids pUC9-TSF11 and pUC9delH3-pTSF-3. These plasmids are derived from pUC9 (Messing and Vieira (1982), Gene 19: 259-268), which contains parts of pBR322.

Commonly used prokaryotic regulatory sequences suitable for constructing plasmid vectors include bacterial promoters for transcription initiation, optionally with an operator, and ribosome binding site sequences. Commonly used promoters include the lactamase (penicillinase) and lactose (lac) promoter systems (Chang et al. (1977), Nature 198: 1056); the tryptophan (trp) promoter system (Goeddel et al. (1980), Nucl. Acids Res. 8: 4057); the lambda-derived P_{L} promoter (Shimatake et al. (1981), Nature 292: 128); and the trp-lac (trc) promoter system (Amann and Brosius (1985), Gene 40: 183).

In addition to bacteria, eukaryotic microbes such as yeast can also be used as hosts. Laboratory strains of Saccharomyces cerevisiae (Baker's yeast) are preferred, although a number of other strains or species are commonly available. Vectors employing, for example, the 2μ origin of replication described in Broach (1983), Meth. Enz. 101: 307, or other yeast compatible origins of replication (see, for example, Stinchcomb et al. (1979), Nature 282: 39; Tschumper et al. (1980), Gene 10: 157; and Clarke et al. (1983), Meth. Enz. 101: 300) can be used. Regulatory sequences for yeast vectors include promoters for the synthesis of glycolytic enzymes (see Hess et al. (1968), J. Adv. Enzyme Reg. 7:149 and Holland et al. (1978), Biochemistry 17: 4900). Additional promoters known in the art include the promoter for 3-phosphoglycerate kinase (Hitzeman et al. (1980), J. Biol. Chem. 255: 2073). Other suitable yeast promoters, which have the additional advantage of transcription controlled by growth conditions and/or genetic background, include the promoter regions for alcohol dehydrogenase 2; isocytochrome C; acid phosphatase; degradative enzymes associated with nitrogen metabolism; the alpha factor system; and enzymes responsible for maltose and galactose utilization.

For yeast hosts, it is also believed that terminator sequences are desirable at the 3′ end of the coding sequences. Such terminators are found in the 3′ untranslated region following the coding sequences in yeast-derived genes.

It is also possible to express nucleic acid sequences in eukaryotic host cell cultures derived from multicellular organisms. See, for example, U.S. Pat. No. 4,399,216 of Axel et al. These systems have the ability to splice out introns, and thus can be used directly to express genomic fragments. For example, a genomic sequence for human cdk4 is found in GenBank record Ace. No. U81031, for pig cdk4 is found in GenBank record Acc. No. U68478, and for X. laevis cdk2 is found in GenBank record Acc. No. U07979, the disclosures of which are herein incorporated by reference. However, non-genomic (e.g., cDNA) sequences can also be expressed.

Useful mammalian host cell lines for expressing mutant cdk2, cdk4 or cdk6 protein according to the present invention include VERO, HeLa, baby hamster kidney (BHK), CV-1, COS (e.g., COS-7), MDCK, NIH 3T3, L, and Chinese hamster ovary (CHO) cell lines. Expression vectors for such cells ordinarily preferably comprise promoters and regulatory sequences compatible with mammalian cells such as, for example, the SV40 early and late promoters (Fiers et al. (1978), Nature 273: 113), or other viral promoters such as those derived from polyoma, Adenovirus 2, bovine papilloma, or avian sarcoma viruses. The controllable promoter hMTII (Karin et al. (1982), Nature 299: 797-802) can also be used.

Depending on the host cell used, transfection of the plasmid vector is accomplished using standard techniques appropriate to the cell. The calcium treatment employing calcium chloride, as described by Cohen (1972), Proc. Natl. Acad. Sci. USA 69: 2110, or the RbCl₂ method described in Maniatis et al., Molecular Cloning: A Laboratory Manual (1982), Cold Spring Harbor Press, p. 254 and Hanahan (1983), J. Mol. Biol. 166: 557-580, can be used for prokaryotes or other cells which contain substantial cell wall barriers. For cells without such cell walls (i.e., eukaryotic; for example mammalian cells), the calcium phosphate precipitation method of Graham and van der Eb (1978), Virology 52: 546, optionally as modified by Wigler et al. (1979), Cell 16: 777-785 can be used. Transformations into yeast can be carried out according to the method of Beggs (1978), Nature 275: 104-109.

Construction of suitable vectors for a given host, containing the desired coding and suitable regulatory sequences, involves standard ligation and restriction techniques which are well understood in the art. Isolated plasmids, nucleic acid sequences, or synthesized oligonucleotides are cleaved, tailored, and re-ligated in the form desired.

The desired nucleic acid coding sequence for insertion into a plasmid vector can be retrieved from available cDNA or genomic DNA libraries, or from available plasmids. Preferred coding sequences are SEQ. ID NO: 1 and SEQ. ID. NO: 2. Alternatively, the desired nucleic acid coding sequence can be synthesized in vitro starting from the individual nucleoside derivatives. For example, nucleic acid sequences of sizeable length, e.g., 500-1000 bp, can be prepared by synthesizing individual overlapping complementary oligonucleotides and filling in single stranded non-overlapping portions using DNA polymerase in the presence of the deoxyribonucleotide triphosphates. This approach has been used successfully in the construction of several genes of known sequence. See, for example, Edge (1981), Nature 292: 756; Nambair et al. (1984), Science 223: 1299; and Jay (1984), J. Biol. Chem. 259: 6311.

Synthetic nucleic acid sequences can be prepared by, for example, the phosphotriester method as described in Edge et al., supra, and Duckworth et al. (1981), Nucl. Acids Res. 9: 1691; or the phosphoramidite method as described in Beaucage and Caruthers (1981), Tet. Letts. 22: 1859 and Matteucci and Caruthers (1981), J. Am. Chem. Soc. 103: 3185. The nucleic acid sequences can also be prepared using commercially available automated oligonucleotide synthesizers.

Typically, synthetic nucleic acids are provided single-stranded sequences. It is often desirable to phosphorylate the 3′ end of the single-stranded nucleic acid sequences prior to annealing with complementary sequences, for example to facilitate linking of nucleic acid fragments to form larger sequences. Phosphorylation of single stranded nucleic acid sequences prior to annealing can be accomplished, for example, using an excess (e.g., approximately 10 units) of polynucleotide kinase to 1 nanomole substrate in the presence of 50 mM Tris, pH 7.6, 10 mM MgCl₂, 5 mM dithiothreitol, 1-2 mM ATP, 0.1 mM spermidine, and 0.1 mM EDTA. If it is desired to radiolabel the nucleic acid sequences, 1.7 pmoles [λ³²P]-ATP (2.9 mCi/mmole) can be added to the reaction cocktail.

Other components for constructing suitable plasmid vectors are available, typically carried in other plasmids. These components can be excised from their source plasmids and ligated together with the nucleic acid sequence of interest, using standard restriction and ligation procedures.

Site specific nucleic acid cleavage, or restriction, is generally performed by treating nucleic acid sequences with suitable restriction enzyme(s) under conditions well-known in the art. Moreover, suitable reaction conditions for a given restriction enzyme are typically specified by the manufacturer of commercially available restriction enzymes. See, e.g., New England Biolabs Product Catalog, 2001.

In general, about 1 microgram of plasmid or nucleic acid sequence is cleaved by one unit of restriction enzyme in about 20 microliters of buffered solution. An excess of restriction enzyme is often used to insure complete digestion of the nucleic acid substrate with incubation times of about one hour to two hours at the optimum temperature for each enzyme as described by the manufacturer.

After each incubation, restriction enzyme can be inactivated and removed from the nucleic acid sequence by extraction with phenol/chloroform, optionally followed by ether extraction, and the nucleic acid recovered from aqueous fraction by precipitation with 2 to 2½ volumes of ethanol. If desired, size separation of the cleaved nucleic acid fragments can be performed by polyacrylamide or agarose gel electrophoresis using standard techniques. A general description of size separation techniques is found in Methods in Enzymology (1980), 65: 499-560.

Many restriction enzymes leave single-stranded overhangs after cleavage of nucleic acid sequences. Nucleic acid fragments with single-stranded overhangs can be ligated with sequences containing complementary overhangs (so called “sticky-end” ligation) or, if there is no overhang, can be ligated with any other blunt-ended nucleic acid sequences.

Nucleic acid fragments can be “blunt-ended” by, for example, incubation with the large fragment of E. coli DNA polymerase I (Klenow fragment) in the presence of the four deoxynucleotide triphosphates (dNTPs), using incubation times of about 15 to 25 min. at 20 to 25° C. in 50 mM Tris pH 7.6, 50 mM NaCl, 6 mM MgCl₂, 6 mM DTT and 0.1-1.0 mM dNTPs. The Klenow fragment fills in 5′ single-stranded overhangs, but “chews back” protruding 3′ single strands. After treatment with Klenow fragment, the reaction mixture containing the blunt-ended nucleic acid fragments is extracted with phenol/chloroform and ethanol precipitated. Treatment under appropriate conditions with S1 nuclease or BAL-31 results in hydrolysis of any remaining single-stranded portions.

Ligation of nucleic acid sequences can be performed in 15-50 microliter volumes under the following standard conditions and temperatures, for example, 20 mM Tris-HCl pH 7.5, 10 mM MgCl₂, 10 mM DTT, 33 microgram/ml BSA, 10 mM-50 mM NaCl, and either 40 micromolar ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are typically performed at 33-100 micrograms/ml total DNA concentrations (5-100 nM total end concentration). Intermolecular blunt end ligations are typically performed at 1 micromolar total ends concentration.

To avoid unwanted self-ligation of the vector, fragments of nucleic acids used for vector construction are commonly treated with bacterial alkaline phosphatase (BAP) or calf intestinal alkaline phosphatase (CIP) in order to remove the 5′ phosphates. Phosphatase reactions are typically conducted at pH 8 in approximately 10 mM Tris-HCl, 1 mM EDTA using about 1 unit of BAP or CIP per microgram of vector, at 60° C. for about one hour. Phosphatased nucleic acid fragments are recovered by extraction with phenol/chloroform and ethanol precipitation as described above.

To verify correct construction of the plasmid vector, plasmids are transfected into a suitable host, amplified, extracted, and analyzed by sequence and/or restriction analysis as is known in the art. For example, any E. coli strain (e.g., MC1061 described in Casadaban et al. (1980), J. Mol. Biol. 138: 179-207) or other suitable host can be transfected with the finished plasmid according to known techniques. Successful transfectants are selected by ampicillin, tetracycline or other antibiotic resistance (or with other appropriate markers), as is understood in the art.

Plasmids can be extracted from the transfectants according to known methods, for example the method of Clewell et al. (1969), Proc. Natl. Acad. Sci. (USA) 62: 1159, optionally following chloramphenicol amplification (see Clewell (1972), J. Bacteriol. 110: 667). See also Holmes et al. (1981), Anal. Biochem. 114: 193-197 and Birnboim et al. (1979), Nucl. Acids Res. 7: 1513-1523. Commercially available nucleic acid “mini-preps” can also be used, such as are available from Quiagen, Boehringer Mannheim, Stratagene, Invitrogen, and others (see DeFrancesco L (1997), The Scientist 11: 22 for a description of commercially available plasmid preparation kits and their suppliers).

Isolated plasmid can be analyzed, for example, by hybridization to appropriate radiolabeled probes in a “dot blot” analysis (e.g., as described by Kafatos et al. (1977), Nucl. Acid Res. 7: 1541-1552); Southern hybridization analysis (e.g., as described by Southern (1975), J. Mol. Biol. 98: 503-517); restriction enzyme analysis; or by nucleic acid sequencing (e.g., via the dideoxy nucleotide method of Sanger et al. (1977), Proc. Natl. Acad. Sci. (USA) 74: 5463, as further described by Messing et al. (1981), Nucl. Acids Res. 9: 309, or the method of Maxam et al. (1980), Methods in Enzymology 65: 499).

Mutants of the cdk2, cdk4 or cdk6 proteins can be directly expressed from plasmids constructed as described above, if the nucleic acid coding sequence in the plasmid already contains sequence modification sufficient to produce the desired amino acid change(s). Alternatively, plasmids containing unmodified cdk2, cdk4 or cdk6 protein coding sequences can be subjected to site-directed mutagenesis to produce the desired sequence changes. Mutant proteins can then be expressed from the mutagenized plasmids.

A preferred method for generating mutants of cdk2, cdk4 or cdk6 proteins that exhibit activating mutations is site-directed mutagenesis. This technique can be used to target precisely the location and type of modification desired; for example, a single base in a nucleic acid sequence can be changed to any of the other three bases by means of oligonucleotide primers. The nucleic acid can then encode a totally different amino acid, resulting in a mutagenized protein (see McPherson, M. J., Directed Mutagenesis, Oxford Univ. Press, NY (1991); Carter, P., Biochem. J. 237: 1-7 (1986); and Nickoloff and Deng (1992), Anal. Biochem. 200: 81-88). Nucleic acid sequences for site-directed mutagenesis can be provided with appropriate regulatory elements suitable for any host, including bacteria, yeast, or eukaryotic cells. Exemplary regulatory elements and hosts are discussed in more detail below.

Techniques for site-directed mutagenesis are well-known in the art. Such techniques generally involve inducing nucleotide base changes or deletions at a specific codon in the coding sequence of interest, using a synthetic primer which omits or alters a codon so that it codes for another amino acid. It is apparent that when deletions are introduced, the proper reading frame for the coding sequence must be maintained for expression of the desired protein.

For example, the methods of Zoller and Smith (1982), Nucl. Acids Res. 10: 6487-6500 or Adelman et al. (1983), DNA 2: 183-193 can be used. These methods involve annealing a synthetic oligonucleotide primer carrying limited base mis-matches to a stretch of single stranded phage DNA carrying the coding sequence to be mutagenized.

The mutagenizing primer can be hybridized to the genome of a single-stranded bacteriophage such as M13 or phi-X174, into which a single-stranded portion of the nucleic acid coding sequence has been cloned. The source of the single-stranded nucleic acid coding sequence is preferably a plasmid vector as described above. It will be appreciated that the phage can carry either the sense strand or antisense strand of the gene. Mutagenizing primers are complementary to the coding strand sequence carried by the phage, except for a limited, preferably single-base, mismatch which defines a desired codon change.

Techniques for designing mutagenizing primers of the appropriate size and sequence to achieve the desired changes are known in the art. For example, relevant factors in designing primers for use in oligonucleotide-directed mutagenesis (e.g., extent of mismatch, overall primer size, size of portions flanking the mutation site, etc.) are described by Smith and Gillam (1981), in Genetic Engineering: Principles and Methods, Plenum Press 3: 1-32. The specific changes to be made in the coding sequence are chosen beforehand, based on sequence and structure-function analyses described above.

In general the overall length of the mutagenizing primer will be such as to optimize stable, selective hybridization at the mutation site, with the 5′ and 3′ extensions from the mutation site being of sufficient size to avoid editing of the mutation by the exonuclease activity of DNA polymerase. Mutagenizing primers in accordance with the present invention can contain from about 18 to about 45 bases, preferably from about 23 to about 27 bases, with at least about three bases extending 3′ from the mutation site.

The mutagenizing primer is hybridized to the phage genome carrying the coding sequence to be altered, for example according to the conditions described by Smith and Gillam, supra. Hybridization temperature can range between about 0° C. and about 70° C., preferably between about 10° C. to about 50° C. After hybridization, the primer is extended on the phage DNA by reaction with DNA polymerase I, T₄ DNA polymerase, or other suitable DNA polymerase. The resulting linear double-stranded DNA is then converted to closed circular double-stranded DNA by treatment with a DNA ligase such as T₄ DNA ligase. Any remaining single-stranded DNA molecules can be destroyed by S1 endonuclease treatment.

The resulting closed circular double-stranded DNA is transfected into a phage-supporting host bacterium. Cultures of the transfected bacteria are plated in top agar, permitting plaque formation from single cells which harbor the phage. Theoretically, 50% of the new plaques will contain phage having, as a single strand, the altered coding sequence, and 50% will have the original coding sequence. Plaques containing the altered coding sequence are hybridized with labeled nucleic acid probe containing a region exactly complementary to the altered sequence. The hybridization conditions are stringent enough to permit only annealing of the probe only to an exact match; mismatches with the original strand are sufficient to prevent probe hybridization. Plaques showing hybridization with the probe are then picked, cultured, and the DNA recovered. The recovered DNA can then be used to express the mutant protein.

Other site-directed mutagenesis methods are available which do not require the generation of single-stranded bacteriophage template, but rather can be performed with double-stranded plasmid DNA. For example, the method of Weiner et al. (1995), Molecular Biology: Current Innovations and Future Trends (Griffin A M and Griffin H G, eds.), Horizon Scientific Press, Norfold, UK can be used. This method involves the use of the polymerase chain reaction (PCR) to incorporate site-specific mutations into virtually any double-stranded plasmid, thus eliminating the need for M13-based vectors or single-stranded rescue. Plasmids as described above comprising cdk2, cdk4 or cdk6 protein coding sequences are suitable for use in this method.

Alterations on the coding sequences can be introduced via one or both PCR primers. Mutagenizing primers can be designed using well-known techniques, based upon a predetermined amino acid change. Generally, primers for PCR-based site-directed mutagenesis will be approximately 12-24 bases long, and have a G-C content of about 45-60%. Preferably, the primers will avoid stretches of A's or T's greater than 3 bases long, and have a G or C as the 3′-most base to act as a stabilizing “clamp” during annealing with the target sequence.

Plasmid DNA strands are separated during the PCR denaturing step, allowing efficient polymerization of the mutagenizing PCR primers to the plasmid template and subsequent extension of the primers around the entire plasmid.

In order to reduce expansion of any undesired mutations, the template concentration is typically increased approximately 1000-fold over conventional PCR conditions, and the number of cycles is reduced from about 25-30 to about 5-10. As the goal is to generate a PCR product encompassing the full length of the plasmid template, heat-stable polymerases capable of synthesizing large fragments (e.g., up to 10 kb) are preferably used. A suitable polymerase is Taq Extender™ from Stratagene.

As Taq DNA polymerases tend to extend the newly synthesized DNA strand beyond the template, the PCR reactions are treated with Pfu DNA polymerase to remove the Taq DNA polymerase-extended base(s) on the linear PCR product.

Also, DNA isolated from almost all common strains of E. coli is Dam-methylated at the sequence 5′-GATC-3′. To remove “parental” DNA containing the unaltered sequence from the reaction mixture, the restriction endonuclease DpnI (which recognizes this sequence where the A residue is methylated) is therefore added after the PCR step. The DpnI treatment digests the methylated parental template and hybrid DNA molecules consisting of a methylated parent strand and a newly synthesized strand. The DpnI- and Pfu-treated linear PCR products are then circularized with T₄ DNA ligase and transfected into an appropriate bacterial host for expression of the altered protein.

A suggested protocol for PCR-based site directed mutagenesis is given in Table 4.

TABLE 4
PCR-based Site Directed Mutagenesis

Plasmid template DNA (approximately 0.5 picomole) is added to a PCR
cocktail containing, in 25 microliter of 1× mutagenesis buffer: (20 mM
Tris HCl, pH 7.5; 8 mM MgCl₂; 40 microgram/ml BSA); 12-20
picomole of each primer (one of which must contain a 5-prime
phosphate), 250 uM each dNTP, 2.5 U Taq DNA polymerase, 2.5 U of
Taq Extender (Stratagene).
The PCR cycling parameters are 1 cycle of: 4 min at 94° C., 2 min at 50
° C. and 2 min at 72° C.; followed by 5-10 cycles of 1 min at 94° C., 2
min at 54° C. and 1 min at 72° C. (step 1).
The parental template DNA and the linear DNA extended from the
mutagenesis primer are treated with DpnI (10 U) and Pfu DNA
polymerase (2.5 U).
The reaction is incubated at 37° C. for 30 min and then transferred to 72°
C. for an additional 30 min (step 2).
Mutagenesis buffer (1×, 115 microliter, containing 0.5 mM ATP) is
added to the DpnI-digested, Pfu DNA polymerase-treated PCR products.
The solution is mixed and 10 microliter is removed to a new microfuge
tube and T₄ DNA ligase (2-4 U) added.
The ligation is incubated for greater than 60 min at 37° C. (step 3).
The treated solution is transformed into competent E. coli (step 4).

Site-directed mutagenesis can also be performed on double-stranded plasmid DNA without using PCR. Such methods are based on the ability of certain DNA polymerases to synthesize entire plasmids using two complementary primers. A kit for performing this method is available from Stratagene (The QuikChange™ kit).

This method uses double stranded plasmids purified, for example, by miniprep or cesium chloride gradient. Plasmid DNA with the unaltered coding sequence of interest are annealed with two synthetic oligonucleotide primers containing the desired mutation. The oligonucleotide primers, each complementary to opposite strands of the vector, are extended during temperature cycling with a high-fidelity, long extending DNA polymerase such as PfuTurbo™ DNA polymerase (Stratagene).

On incorporation of the oligonucleotide primers, a mutated plasmid containing staggered nicks is generated by primer extension. The reaction mixture is then treated with DpnI to digest methylated parental DNA template and hemi-methylated hybrid DNA, thus selecting for the mutagenized plasmid. The nicked vector DNA incorporating the desired mutations is then transformed into a suitable E. coli host for expression, for example “XL1-Blue” or “XL10-Gold” cells. Materials and methods for performing the QuikChange method are available from the manufacturer.

In addition to predetermined alterations in the cdk2, cdk4 or cdk6 proteins, libraries of plasmids expressing a spectrum of random mutations can be produced. One technique for producing such libraries is termed “error-prone PCR.” In this method, random mutations are deliberately introduced into nucleic acid coding sequences during PCR through the use of error-prone DNA polymerases. The mutated DNA sequences are cloned into expression vectors, and the resulting libraries of mutant proteins are screened for the desired protein activity. Unaltered nucleic acid coding sequences suitable for use in this method can be linear (preferably double-stranded) nucleic acid molecules comprising all or part of the cdk2, cdk4 or cdk6 protein coding sequences, or can be plasmids containing these sequences prepared as described above.

Error-prone PCR methods commonly employ Taq DNA polymerase, as it lacks proofreading activity and is inherently error prone. A preferred Taq DNA polymerase is the Mutazyme™ DNA polymerase available from Stratagene. To achieve useful mutation frequencies, the error rate of Taq DNA polymerase can be further increased by employing PCR reaction buffers containing Mn⁺⁺ and unbalanced dNTP concentrations.

Different mutation frequencies can be achieved by varying the DNA template concentration. A low initial template concentration means that the target sequence undergoes greater amplification in a given number of PCR cycles. As the Taq polymerase error rate is based on the degree of target sequence amplification, higher mutation frequencies are achieved by lower initial template concentrations. Conversely, lower mutation frequencies are achieved by using higher initial template concentrations. For structure-function analyses, a low mutation rate (1-3 nucleotide changes, or 1 amino acid change per kilobase coding sequence) is typically used. In directed evolution studies, medium mutation frequencies (3-7 nucleotide changes, or 1-4 amino acid changes per kilobase coding sequence) are generally employed. Proteins with the desired activity can also be isolated from highly mutagenized libraries exhibiting 7-20 point mutations per kilobase coding sequence.

Appropriate amounts of target nucleic acid needed to achieve the desired mutation frequency (MF) are given in Table 5, adapted from Cline and Hogrefe “Randomize Gene Sequences with New PCR Mutagenesis Kit,” Stratagene (2001), 13(4): 157-162, the entire disclosure of which is herein incorporated by reference.

TABLE 5
Initial Amounts of Template DNA for Desired Mutational Frequency

| Initial template | | | |
|---|---|---|---|
| amount^{b} | | approximate # mutations per kb | Mutation Level |
| 100 | ng | 1-2.5 | low |
| 10 | ng | 2-4 | |
| 1 | ng | 3-4.5 | medium |
| 100 | pg | 4-5.5 | |
| 10 | pg | 5-6.5 | |
| 1 | pg | 6-7 | |
| Double | PCR | 7-13 | high |
| Triple | PCR | 9-20 | |

^{b}amount of mutational target; determined as: (size of mutational target/size of plasmid DNA) x (plasmid DNA template amount).

A kit and method for performing error prone PCR (Genemorph™) is available from Stratagene.

Mutant cdk2, cdk4 or cdk6 proteins with the desired biological activity can be identified by their ability to reversibly induce continual cell growth when added to cells in culture. For example, a mutated protein is added to a viable culture of mouse embryonic fibroblasts isolated and cultured according to the technique of Todaro and Green (1963), J. Cell. Biol. 17: 299-313. The cultured fibroblasts are then evaluated for certain morphological and biochemical changes indicative of an immortalized phenotype. Such changes including a decreased doubling time, a higher proportion of cells in the S and G2/M phases than controls, and no contact growth inhibition. This fibroblast immortalization assay is described in Example 4 below.

The continual growth-inducing compositions of the invention can additionally comprise a compound comprising the catalytic subunit of telomerase, or a biologically active derivative, homolog or analog thereof.

The catalytic subunit of telomerase, also called TERT, is a protein of 1132 amino acids that exhibits reverse transcriptase activity. Compounds comprising TERT can be expressed from nucleic acid sequences known in the art, using the techniques described above. For the purposes of the present invention, the TERT protein should retain its natural biological activity; i.e., the ability to catalyze RNA-dependent elongation of the 3′ terminus of a chromosome, and thus maintain telomere length above the critical threshold. One of ordinary skill in the art can readily determine whether a compound comprising an TERT protein, or a fragment, derivative, homolog or analog thereof, is biologically active by testing the compound for telomerase activity by known methods; for example, by the telomeric repeat amplification protocol (TRAP) assay as described Kim et al. (1994), Science 266: 2011-2015, the disclosure of which is herein incorporated by reference. Identification of TERT proteins with the TRAP assay is also disclosed in Example 9 below.

As used herein, “TERT protein” includes the TERT protein from any species. TERT nucleic acid sequences which can be used for expressing TERT proteins are given in Table 6 below One of ordinary skill can readily identify nucleic acid sequences from other species as encoding TERT proteins, based on similarity to the sequences listed in Table 6. Nucleic acid sequences that exhibit substantial similarity to the Table 6 sequences can be considered as encoding hTERT proteins. Extant proteins can also be identified as TERT proteins by comparison to the protein sequences listed in Table 6.

TABLE 6
TERT cDNA and Protein Sequences

| | | |
|---|---|---|
| | GenBank | |
| Species | Acc. No.1 | SEQ ID NO: |

| | | | |
|---|---|---|---|
| Xenopus laevis | AAG43537 | protein | 56 |
| (African clawed frog) | | | |
| Mesocricetus auratus | AAF17334 | protein | 57 |
| (golden hamster) | | | |
| Rattus norvegicus | AF247818 | protein | 58 |
| (rat) | | cDNA | 59 |
| Mus musculus | AAC09323 | protein | 60 |
| (mouse) | | | |
| Homo sapiens | NP_003210 | protein | 61 |
| Cryptosporidium parvum | AY034376 | protein | 62 |
| | | cDNA | 63 |
| Giardia intestinalis | AF195121 | protein | 64 |
| | | cDNA | 65 |
| Mus musculus | AF157502 | protein | 66 |
| (mouse) | | cDNA | 67 |
| Candida albicans | AF216872 | protein | 68 |
| (yeast) | | cDNA | 69 |

¹GenBank records identified in Table 6 are herein incorporated by reference.

When used as part of the continual growth-inducing composition of the present invention, the TERT proteins, or fragments, derivatives, homologs or a analogs thereof, are preferably present in equimolar amounts with the cdk2, cdk4 or cdk6 compounds described above.

The compounds comprising the continual growth-inducing composition of the invention, including the TERT compounds described above, can also comprise one or more modifications that allow transport of the compound into a cultured cell, when contacted with the cell in culture. For example, the compounds can be modified with a leader peptide sequence that directs entry of the compound into the cell, when the compound is administered exogenously in culture. Such leader sequences, also known as “protein transduction domains” or “PTDs,” are well known in the art. A PTD can be located anywhere on the continual growth-inducing compound that does not disrupt the compound's biological activity. For compounds comprising peptides, the PTD is preferably located at the N-terminal end.

It is known that bioactive, exogenous proteins and other molecules can be delivered to any mammalian cell type by linking it to a PTD. This technique is known as “protein transduction.” See Schwarze et al. (1999), Science 285: 1569-1572, the entire disclosure of which is herein incorporated by reference. Proteins ranging in size from 15 to 120 kD have been transduced into a wide variety of human and murine cell types in vitro using this method. See Nagahara et al. (1998), Nature Med. 4: 1449; Ezhevsky et al. (1997), Proc. Natl. Acad. Sci. U.S.A. 94: 10699; Lissy et al. (1998), Immunity 8: 57; and Gius et al. (1999), Cancer Res. 59: 2577, the entire disclosures of which are herein incorporated by reference.

Without wishing to be bound by a particular theory, entry of exogenously added, PTD-linked compounds into the cell during protein transduction appears to occur in a rapid, concentration-dependent fashion. Moreover, the process appears to be receptor and transporter independent, see Derossi et al. (1996), J. Biol. Chem. 271: 18188, and may directly involve the lipid bilayer component of the cell membrane. Thus, all cell types, in particular mammalian cell types, are susceptible to protein transduction. Exogenous administration of PTD-linked compounds to cultured cells results in the rapid delivery of a roughly equal amount of the compound to each cell.

The PTD can comprise any of the known PTD sequences including for example, a peptide of eleven arginine residues (SEQ ID NO: 70) or the NH₂-terminal 11-amino acid protein transduction domain from the human immunodeficiency virus TAT protein (SEQ ID NO: 71) Other suitable leader sequences include, but are not limited to, other arginine-rich sequences; e.g., 9 to 11 arginines, or six or more arginines in combination with one or more lysines or glutamines. Such leader sequences are know in the art; see, e.g., Guis et al. (1999), Cancer Res. 59: 2577-2580, the disclosure of which is herein incorporated by reference.

Preferably, the PTD is designed so that it is cleaved from the continual growth-inducing compound within the cell. Amino acid sequences susceptible to enzymatic cleavage within a cell are known in the art.

The PTD can also comprise a label (e.g., substances which are magnetic resonance active; radiodense; fluorescent; radioactive; detectable by ultrasound; detectable by visible, infrared or ultraviolet light) so that entry of the PTD-linked compound into the cells can be monitored. Suitable labels include, for example, fluorescein isothiocyanate (FITC); peptide chromophores such as phycoerythrin or phycocyanin and the like; bioluminescent peptides such as the luciferases originating from Photinus pyrali; fluorescent proteins originating from Renilla reniformi; and radionuclides such as P³², P³³, S³⁵, I¹²⁵ or I¹³¹. For example, the label can comprise an NH₂-terminal fluorescein isothiocyanate (FITC)-Gly-Gly-Gly-Gly motif that is conjugated to the PTD.

Methods of modifying PTD sequences with labels are well known to those skilled in the art. For example, methods of conjugating fluorescent compounds such as fluorescein isothiocyanate to short peptides are described in Danen et al., Exp. Cell Res., 238:188-86 (1998), the entire disclosure of which is herein incorporated by reference. Methods of producing a FITC-labeled PTD leader sequence are described in Schwarze et al. (1999), Science 285: 1569-1572 the disclosure of which is herein incorporated by reference. Methods of radiolabeling peptides with radionuclides such as ¹²⁵I and ³⁵S are disclosed by Sambrook et al. in Molecular Cloning: A Laboratory Manual, Cold Spring Harbor Laboratories, Second Ed., (1989), pp. 18.24-18.29, the entire disclosure of which is herein incorporated by reference.

It is understood that the compounds comprising the continual growth-inducing composition can themselves be modified with a label, as described above for the PTD sequence.

The compounds comprising the continual growth-inducing composition and the PTD sequence can be linked by any means which allows formation of a covalent bond between the PTD sequence and the compounds. Such methods are known to those of ordinary skill in the art. For example, fusion peptides comprising a compound and a PTD can be generated by any means which permits linking two or more peptide sequences, including standard recombinant nucleic acid techniques, or solid phase peptide synthesis techniques.

Kits and methods for producing fusion peptides comprising a PTD linked to a protein of interest are commercially available. For example, the TransVector™ system (Q-B10 gene) produces fusion proteins comprising the 16 amino acid Penetratin™ peptide leader sequence, which corresponds to the Drosophila antennapedia DNA binding domain. The Voyager™ system allows creation of fusion peptides comprising the V22 PTD from Herpes Simplex Virus-1. One of ordinary skill in the art is able to generate continual growth-inducing compounds of the invention linked to a suitable PTD using such kits and methods.

Alternatively, the continual growth-inducing compositions of the invention can be introduced into a cultured cell by fusion of a liposome encapsulating the compositions with the cell membrane. Techniques to encapsulate proteins in a liposome for delivery into a cell are well known in the art. For example, the ProVectin™ Protein Delivery Reagent, available from Ingenex, can be used to deliver the present compounds into cultured cells. It is understood that the compounds comprising continual growth-inducing compositions administered to cells by liposome encapsulation do not require PTDs. For purposes of the invention, encapsulation of the continual growth inducing compounds is a “modification which allows entry of the compositions into a cell.”

The compounds comprising the continual growth-inducing compositions of the invention can comprise natural or synthetic peptides produced by any known means, including synthesis by biological systems and chemical methods.

Biological synthesis of peptides is well known in the art, and includes the transcription and translation of a synthetic gene encoding cdk2, cdk4, cdk6 proteins with an activating mutation, or biologically active fragments, homologs, and derivatives thereof. Chemical peptide synthesis includes manual and automated techniques well known to those skilled in the art.

For example, automated peptide synthesis can be performed with commercially available peptide synthesizers. Biologically active fragments according to the invention can also be obtained by the digestion or fragmentation of larger natural or synthetic peptides. Techniques to synthesize or otherwise obtain peptides and peptide fragments are well known in the art.

The peptides and fragments comprising the compounds of the present invention can be synthesized de novo using conventional solid phase synthesis methods. In such methods, the peptide chain is prepared by a series of coupling reactions in which the constituent amino acids are added to the growing peptide chain in the desired sequence. The use of various N-protecting groups, e.g., the carbobenzyloxy group or the t-butyloxycarbonyl group; various coupling reagents e.g., dicyclohexylcarbodiimide or carbonyldimidazole; various active esters, e.g., esters of N-hydroxyphthalimide or N-hydroxy-succinimide; and the various cleavage reagents, e.g., trifluoroactetic acid (TFA), HCl in dioxane, boron tris-(trifluoracetate) and cyanogen bromide; and reaction in solution with isolation and purification of intermediates are methods well-known to those of ordinary skill in the art.

A preferred peptide synthesis method follows conventional Merrifield solid phase procedures well known to those skilled in the art. Additional information about solid phase synthesis procedures can be had by reference to Steward and Young, Solid Phase Peptide Synthesis, W. H. Freeman & Co., San Francisco, 1969; the review chapter by Merrifield in Advances in Enzymology 32:221-296, F. F. Nold, Ed., Interscience Publishers, New York, 1969; and Erickson and Merrifield, The Proteins 2:61-64 (1990), the entire disclosures of which are herein incorporated by reference. Crude peptide preparations resulting from solid phase syntheses can be purified by methods well known in the art, such as preparative HPLC. The amino-terminus can be protected according to the methods described for example by Yang et al., FEBS Lett. 272:61-64 (1990), the entire disclosure of which is herein incorporated by reference.

The compounds comprising the continual growth-inducing composition of the present invention include derivatives of cdk2, cdk4 or cdk6 proteins having an activating mutation. The techniques for obtaining these derivatives are known to persons having ordinary skill in the art and include, for example, standard recombinant nucleic acid techniques, solid phase peptide synthesis techniques and chemical synthetic techniques as described above. Linking groups can also be used to join or replace portions of cdk2, cdk4, cdk6 and other peptides. Linking groups include, for example, cyclic compounds capable of connecting an amino-terminal portion and a carboxyl terminal portion of cdk2, cdk4, or cdk6. Techniques for generating derivatives are also described in U.S. Pat. No. 6,030,942 the entire disclosure of which is herein incorporated by reference (derivatives are designated “peptoids” in the U.S. Pat. No. 6,030,942 patent). Derivatives can also incorporate labels such as are described above into their structure.

Examples of derivatives according to the present invention include, for example, synthetic variants of cdk2, cdk4 or cdk6 proteins having an activating mutation. Derivatives can also include, for example, fusion peptides in which a portion of the fusion peptide has a substantially similar amino acid sequence to cdk2, cdk4 or cdk6 proteins having an activating mutation. Such fusion peptides can be generated as described above.

The compounds comprising the continual growth-inducing compositions of the invention also include homologs of cdk2, cdk4 or cdk6 proteins having an activating mutation. Homologs have substantially similar amino acid sequence to cdk2, cdk4, cdk6 proteins and can be identified on this basis.

The compounds of the invention also include analogs of cdk2, cdk4 or cdk6 proteins having an activating mutation. The analogs of the invention can, for example, be small organic molecules capable of binding the D-type cyclins and phosphorylating the Rb proteins, or small organic molecules capable of binding cyclins A or E. Analogs can incorporate labels such as are described above into their structure.

Without wishing to be bound by a particular theory, it is believed that the present analogs comprise a structure, called a pharmacophore, that mimics the physico-chemical and spatial characteristics of cdk2, cdk4 or cdk6 proteins having an activating mutation. Consequently, pro-analogs can, for example, be designed based on variations in the molecular structure of the cdk2, cdk4 or cdk6 protein active sites or portions of cdk2, cdk4 or cdk6 proteins. The structure and probable function of the various portions of the cdk2, cdk4 or cdk6 proteins can be determined, for example, using nuclear magnetic resonance (NMR), crystallographic, or computational methods which permit the electron density, electrostatic charges or molecular structure of these peptides to be mapped, as discussed above.

Alternatively, pro-analogs of a cdk2, cdk4 or cdk6 proteins having an activating mutation can be designed, for example, by using the retrosynthetic, target-oriented, or diversity-oriented synthesis strategies described by Schreiber (2000), Science 287:1964-1969, the entire disclosure of which is herein incorporated by reference. Retrosynthetic strategies, for example, require that key structural elements in a molecule such as cdk2, cdk4 or cdk6 protein having an activating mutation be identified and then incorporated into the structure of otherwise distinct pro-analogs generated by organic syntheses. U.S. Pat. No. 6,030,942, in particular Example 4 therein, describes retrosynthetic methods for the design and selection of analogs based on key structural elements in an inhibitory peptide, and is herein incorporated by reference in its entirety (analogs are designated “peptidomimetics” in the 6,030,942 patent).

The solid-phase synthesis methods described by Schreiber supra can be used to generate a library of distinct pro-analogs generated by organic syntheses. Briefly, a suitable synthesis support, for example a resin, is coupled to a pro-analog precursor. The pro-analog precursor is subsequently modified by organic reactions such as, for example, Diels-Alder cyclization. The immobilized pro-analog can then be released from the solid substrate. Pools and subpools of pro-analogs can be generated by automated synthesis techniques in parallel, such that all synthesis and resynthesis can be performed in a matter of days. Such pools and subpools of pro-analogs are said to comprise libraries. Once generated, pro-analog libraries can be screened for analogs; i.e. compounds exhibiting one or more biological activities of cdk2, cdk4 or cdk6 proteins having an activating mutation. Analogs can thus be identified by their ability to reversibly induce continual cell growth as determined, for example, by the cell culture assay described in Example 4 below.

The continual growth-inducing compounds of the invention can additionally comprise a TERT protein, or a biologically active fragment, derivative, homolog or analog thereof. Biologically active fragments, derivatives, homologs or analogs of TERT proteins can be made as discussed above for cdk2, cdk4 and cdk6 proteins. One of ordinary skill in the art can readily determine if a fragment, derivative, homolog or analog of TERT protein is biologically active by the ability of the compound to extend or maintain telomeric sequences, for example as measured by the telomeric repeat amplification protocol (TRAP) assay described in Example 9 below.

The present invention provides methods of inducing continual growth in viable cultures of eukaryotic cells by contacting the cultured cells with an effective amount of a continual growth-inducing composition. The continual growth-inducing composition comprises at least one of the cdk2, cdk4, or cdk6 proteins having an activating mutation, or biologically active fragments, derivatives, homologs or analogs thereof, as described above. The cells experience a state of continual growth for as long as they are in contact with the continual growth-inducing composition. Optionally, the composition further comprises a TERT protein, or a biologically active fragment, derivative, homolog or analog, as described above.

Methods of contacting cultured cells with exogenous compositions are known in the art, and include administering a composition either directly to the cells, or in the culture media. Preferably, an effective amount of a continual growth-inducing composition is included in the fresh growth media which is periodically given to cells growing in culture. For example, a concentrated solution of the continual growth-inducing composition in a carrier such as sterile water, saline, growth media or the like can be diluted into fresh growth media prior to applying the fresh growth media to the cultured cells.

Any eukaryotic cell type that can be placed in culture in a viable state, for a time sufficient to allow contact with an effective amount of a continual growth-inducing composition, can be used in the present methods. Suitable culturable cell types include cells obtained from amphibians, reptiles, birds, mammals, fish, arthropods, and insects. Preferred cells are those obtained from mammals, for example humans; rodents (e.g., mice, rats and guinea pigs); rabbits; ovine mammals (e.g., sheep and goats); bovine mammals (e.g., cows); and porcine mammals (e.g., pigs). Methods for obtaining and culturing such cells are well-known in the art.

In one embodiment, the invention provides methods for inducing a state of continual growth in stem cells derived from an animal; for example, in hematopoietic stem cells, embryonic stem (ES) cells or normal blastocyst cells. Methods of obtaining embryonic stem cells and normal blastocyst cells are known in the art; see for example Thomson et al., (1998) Science 282:1145-47; and Reubinoff et al. (2000) Nat. Biotechnol. 18: 399-404, the entire disclosures of which are herein incorporated by reference. The induction of a continual state of growth in stem cells by the present methods does not lead to terminal differentiation of the stem cells. Thus, large numbers of stem cells can be produced for use in research, transplantation, etc.

Murine ES cell lines suitable for use in the present methods include the AB-1 line grown on mitotically inactive SNL76/7 cell feeder layers (McMahon and Bradley (1990), Cell 62: 1073) essentially as described (Robertson, E. J. (1987) in Teratocarcinomas and Embryonic Stem Cells: A Practical Approach. E. J. Robertson, ed. (Oxford: IRL Press), p. 71-112). Other suitable murine ES lines include, but are not limited to, the E14 line (Hooper et al. (1987) Nature 326: 292-295), the D3 line (Doetschman et al. (1985) J. Embryol. Exp. Morphj. 87: 27-45), and the CCE line (Robertson et al. (1986) Nature 323: 445-448).

An extensive list of human and murine stem cell lines suitable for use in the present methods are listed in Appendix D of “Stem Cells: Scientific Progress and Future Research Directions,” report of the National Institutes of Health, 2001, the entire disclosure of which is herein incorporated by reference. The stem cells listed in the NIH report, Appendix D include hematopoietic stem cells; mesenchymal stem cells; neural stem cells; neural progenitor cells; ES cells; embryonic primordial germ cells (endoderm, mesoderm and ectoderm); skeletal muscle satellite cells; and blastocyst inner cell masses. Methods for isolating and maintaining these cells in culture are known in the art, as referenced in the NIH report, supra, Appendix D.

The effective amount of continual growth-inducing composition will vary from cell type to cell type. One of ordinary skill in the art is able to determine the effective amount for a given cell type, for example by contacting the cells with increasing concentrations of the composition and observing the morphological and growth characteristics of the cells, as outlined in Example 4 below. The concentration at which the cells exhibit the well-known characteristics of an immortalized phenotype is considered to be the minimum effective amount.

When more than one compound comprises the continual growth-inducing composition, it is preferred that the compounds are present in equimolar amounts. For most cell types, the concentration of the compounds comprising an effective amount of the continual growth-inducing composition is at most 50 nM, preferably at most 100 nM, more preferably at most 150 nM, and particularly preferably at most 300 nM. It is contemplated, however, that the compounds can be present in varying amounts relative to each other, consistent with inducing continual growth in the cultured cells.

When the cultured cells are no longer in contact with the continual growth-inducing composition, the induction of continual growth will cease. The cessation of continual growth will not be immediate, but will rather depend on the kinetics of inactivation or clearance of the continual growth-inducing composition from the cultured cells. Continual growth can be resumed, if desired, by again contacting the cells with the continual growth-inducing composition.

The present invention also provides methods of evaluating potential cancer-causing agents, such as ionizing radiation, mutagens, teratogens, carcinogens, oncogenes and the like. The method comprises contacting cultured cells reversibly induced to exhibit continual growth with potential cancer-causing agents.

As used herein, the step of contacting cultured cells with a potential cancer-causing agent includes administration of such agents exogenously in culture (e.g., by placing a chemical carcinogen, teratogen or mutagen in the culture media); the external application of ionizing radiation (e.g., by irradiation with an irradiator using ¹³⁷cesium as a source; brachytherapy devices, etc.); internal application of ionizing radiation (e.g., delivery of tritium or high-energy radionuclides to the cytoplasm or cell nucleus); or introduction of genetic material into the cell (e.g., transfection with plasmids comprising putative oncogenic sequences, infection with recombinant viruses, etc.).

For example, mutagens such as 9,10-di-methyl-1,2-benz[a]anthracene (DMBA) or tumor promoters such as 12-O-tetradecanoylphorbol-13-acetate (TPA) can be added to the culture medium in varying concentrations to determine the susceptibility of a given cell type transformation. Compounds with undetermined cancer-causing activity can also be screened with the continual growth-induced cells of the invention, in order to evaluate the carcinogenicity of the compounds.

The oncogenic potential of nucleic acid sequences can also be evaluated in the continual growth-induced cells. For example, cultured cells can be transfected with plasmids comprising gene sequences expressing suspected oncogenes, or gene sequences that carry naturally or artificially produced mutations. Methods of transfecting eukaryotic cells with nucleic acid sequences are also well known in the art, and include, for example, direct injection into the nucleus or pronucleus; electroporation; liposome transfer (e.g., with N-[1-(2,3-dioleoyloxy)propyl]-N,N,N-trimethylammonium methylsulfate, also called DOTAP); and calcium phosphate precipitation.

The transforming potential of the cancer-causing agents can be evaluated by observing the growth characteristics and morphology of the cultured cells. Transformed cells will continue to be insensitive to contact-induced growth inhibition even when the continual growth inducing compound is removed (i.e., continual growth is not reversible), and the cells will form foci in the culture vessel when cultured for extended periods. Transformed cells also exhibit characteristic morphological changes, disorganized patterns of colony growth and acquisition of anchorage-independent growth. Transformed cells also have the ability to form invasive tumors in susceptible animals, which can be assessed by injecting the cells, for example, into athymic mice or newborn animals of the same species using techniques well-known in the art.

One of ordinary skill in the art is thus able to identify a transformed phenotype in the cultured cells, and/or determine whether the cells have acquired the ability to form tumors in vivo. See, for example, Combes et al. (1999), “Cell Transformation Assays as Predictors of Human Carcinogenicity: The Report and Recommendations of ECVAM Workshop 39,” ATLA 27, 745-767, the entire disclosure of which is herein incorporated by reference.

The invention will now be illustrated with the following non-limiting examples.

### EXAMPLE 1

### Activating Mutations in the cdk4 Protein Lead to Increased Cell Proliferation and Shorter Cell Cycle

Mouse embryonic fibroblasts (MEFs) cultures were obtained from wild type mice (cdk4^{+/+}), transgenic mice heterozygous for the cdk4^{R24C} mutation (cdk4^{+/R24C}), and transgenic mice homozygous for the cdk4^{R24C} mutation (cdk4^{R24C/R24C}), as described in Todaro and Green (1963), J. Cell. Biol. 17: 299-313, the disclosure of which is herein incorporated by reference. The transgenic mice were generated as described in Rane et al. (1999), Nat. Gen. 22: 44-52, the disclosure of which is herein incorporated by reference. The MEFs were propagated in Dulbecco's Modified Eagle Medium (DMEM) supplemented with 10% fetal bovine serum (FBS).

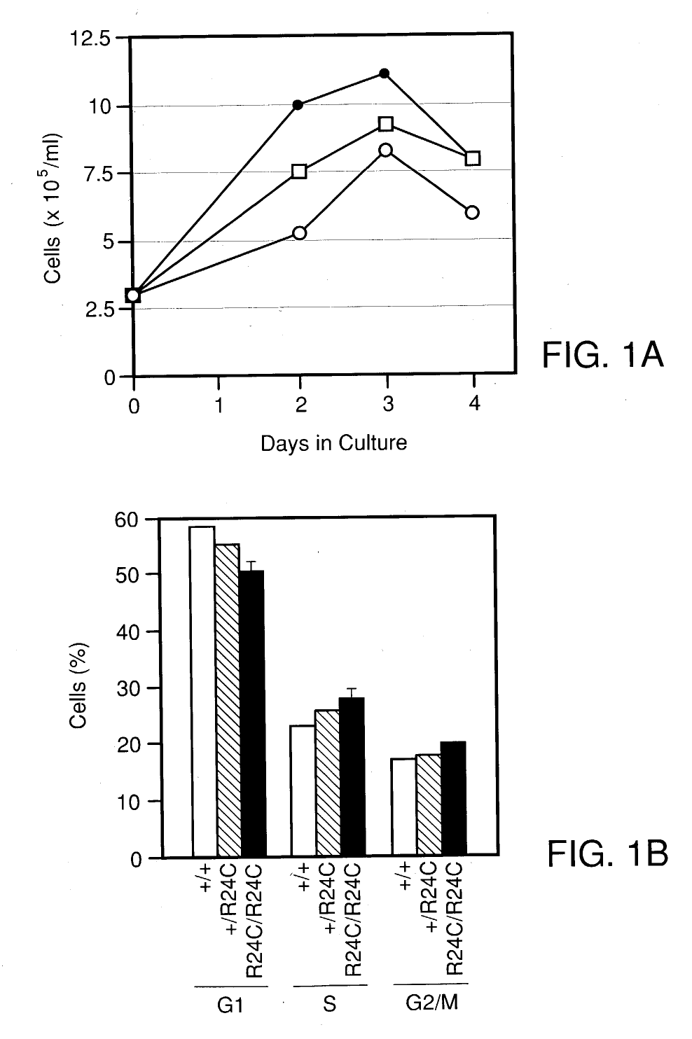

MEFs (passage<4) were used for growth curve and cell cycle phase analyses. For analysis of growth curves, 3×10⁵ cells were plated in 10×2 cm, tissue culture treated polystyrene dishes (Falcon® #353003; Becton Dickinson) and the number of viable cells were counted using trypan blue exclusion analysis for four days. For analysis of cell cycle phases, 3×10⁵ cells were exponentially cultured for 24 hours in 10×2 cm, tissue culture treated polystyrene dishes (Falcon® #353003; Becton Dickinson), after which the cells were fixed in ethanol at −20° C. The percentage of cells in G0/G1, S and G2/M were determined by fluorescence-assorted cytometry (FACS) analysis upon staining for 30 min with propidium iodide after treatment with RNAse A. The results are shown in FIGS. 1a and 1b.

The results show that cdk4^{R24C/R24C} cells grew well in culture and displayed decreased doubling times, indicating that homozygous expression of the cdk4^{R24C} protein caused acceleration of cell proliferation (FIG. 1a). The cdk4^{+/R24C} MEFs exhibited doubling times intermediate between the wild-type and cdk4^{R24C/R24C} MEFs.

To analyze the effects of cdk4^{R24C} mutation on cell cycle progression, a cell cycle analysis was performed using flow cytometry techniques. Exponentially growing cdk4^{R24C/R24C} MEFs exhibited a slightly higher proportion of cells in the S and G2M phases than either the wild-type or cdk4^{+/R24C} MEFs (FIG. 1b).

The consistent increase in the number of cdk4^{R24C/R24C} cells in the S and G2M phases, along with a concomitant decrease in the 2n population of cells (G0/G1), together with their faster growth rate (see FIG. 1a), indicates that the cdk4^{R24C/R24C} MEFs proliferate faster than the wild-type and cdk4^{+/R24C} MEFs. This seems to be due to a decrease in the lengths of the G1-phase, which results in a shorter doubling time.

As the cdk4^{+/R24C} and cdk4^{R24C/R24C} MEFs used in this Example contained one or two copies of an endogenous mutant cdk4 gene, respectively, the induction of continual growth in these cells was not reversible.

### EXAMPLE 2

### Mouse Embryonic Fibroblasts from a Transgenic cdk4^{R24C/R24C} Mouse Fail to Undergo Senescence and Exhibit an Immortalized Phenotype in Culture

Mouse embryonic fibroblasts (“MEFs”) were obtained from wild type mice (cdk4^{+/+}), transgenic mice heterozygous for the cdk4^{R24C} mutation (cdk4^{+/R24C}), and transgenic mice homozygous for the cdk4^{R24C} mutation (cdk4^{R24C/R24C}) as in Example 1. The MEFs were propagated in DMEM media supplemented with 10% FBS according to the 3T3 protocol (Todaro and Green, supra).

For each cell type, 3×10⁵ cells were plated per 10×2 cm, tissue culture treated polystyrene dishes (Falcon® #353003; Becton Dickinson). After 3 days, cells were trypsinized and 3×10⁵ cells from a given dish were re-plated, which constituted one passage. This process was continued for 20 successive passages and the cumulative increase in cell number was calculated according to the formula Log_{(Nfinal/Ninitial)}/Log 2, where Nfinal and Ninitial are the final and initial numbers of cells plated and counted after 3 days, respectively.

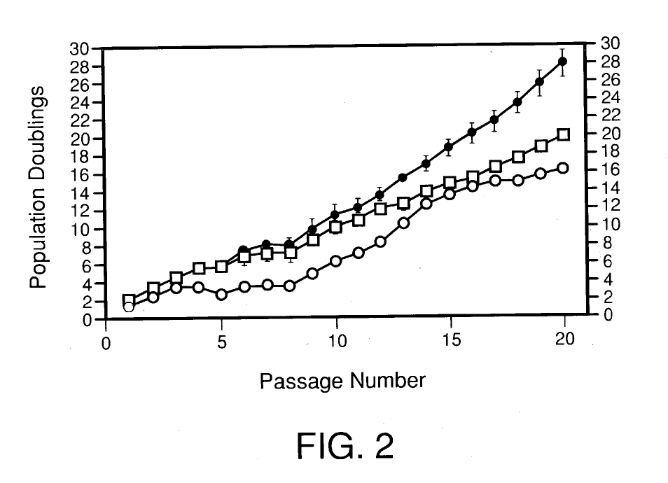

It is known that wild-type MEFs stop dividing after 15-30 generations, and undergo replicative senescence when cultured using the 3T3 protocol (Todaro and Green, supra). As can be seen in FIG. 2, the cdk4^{+/+} cells showed a decline in the proliferation rate at about 8-10 passages, then a short growth spurt between 10-15 passages, followed by the onset of senescence. The cdk4^{+/+} cells underwent approximately 16 population doublings during the first 20 passages in culture (FIG. 2).

Induction of senescence was confirmed by examination of cells by microscopy, where morphological changes associated with a senescence phenotype (flat, large non-dividing cells) were observed. To further confirm the phenotype of replicative senescence in the cdk4^{+/+} cells, the activity of endogenous Senescence Associated Beta-Galactosidase (SA-B-Gal), a specific biomarker of senescence, was examined as described previously (Dimri et al. (1995), Proc. Natl. Acad. Sci. USA 92:9363-7, the entire disclosure of which is herein incorporated by reference). The cdk4^{+/+} cells showed evidence of accumulated SA-B-Gal, suggestive of senescing cells.

In contrast, cdk4^{R24C/R24C} MEFs maintained constant proliferation rates and failed to display any morphological features of senescing cells as evidenced by a failure to accumulate SA-B-Gal. The cdk4^{R24C/R24C} MEFs underwent approximately 30 population doublings after 20 passages in culture (compared to 16 population doublings in control cells), indicative of a significant escape from replicative senescence.

The cdk4^{+/R24C} MEFs displayed an intermediate phenotype, with majority of the cells maintaining a constant rate of proliferation with approximately 20 population doublings in 20 passages. Approximately 50-60% of cdk4^{+R24C} MEFs displayed morphological features of senescent cells. The difference in the behavior of cdk4^{+/R24C} and cdk4^{R24C/R24C} MEFs suggests that induction of continual growth may be dependent on the levels of the mutant cdk4^{R24C} protein inside the cell. Because the cdk4^{+/R24C} and cdk4^{R24C/R24C} MEFs used in this Example contained one or two copies of an endogenous mutant cdk4 gene, respectively, the induction of continual growth in these cells was not reversible.

### EXAMPLE 3

### Preparation of Continual Growth Inducing Composition Comprising cdk4^{R24C}

A nucleic and sequence encoding cdk4 and the NH₂-terminal 11-amino acid PTD from the human immunodeficiency virus TAT protein (SEQ ID NO: 71) are cloned, in the same reading frame, pET-15b expression vector (Novagen) via the multicloning site by standard techniques. This vector allows production of a fusion protein with an N-terminal 6×His tag upon induction with isopropyl-thio-beta-D-thiogalactoside (IPTG). The pET-15b plasmid construct is used to transfect high-expressing bacterial strain BL21 (DE3) (Novagen) according to the manufacturer's protocols; see Novagen's “pET System Manual (9^{th} Ed.)”, May 2000, the disclosure of which is herein incorporated by reference.

One liter of transfected BL21 (DE3) cultures is induced to express the cdk4^{R24C}/PTD/His tag-fusion protein by the addition of IPTG at a final concentration of 1 mM followed by incubation at 37° C. with shaking. Cells are pelleted by centrifugation, resuspended in lysis buffer (8M urea, 100 mL NaCl, 20 mM HEPES (pH=7.0), 20 mM imidazole) and sonicated. Cell lysate is cleared and subjected to Ni-NTA (Qiagen) column chromatography according to the manufacturer's instructions, which binds the 6×His portion of the fusion protein and allows for cdk4^{R24C}/PTD/6×His-fusion protein isolation. After elution from the Ni-NTA column, the fusion protein is dialyzed against 20 mM Hepes/150 mM NaCl according to the procedure of Ezhevsky et al. (1997), Proc. Natl. Acad. Sci. USA 94: 10699-10704, the disclosure of which is herein incorporated by reference. Concentration of the dialyzed fusion protein is determined by the Bio-Rad Protein Determination Assay® (Bio-Rad) and adjusted to 1-10 micrograms/ml before filter sterilization through a 0.02 micron filter.

### EXAMPLE 4

### Reversible Induction of Continual Growth in Cultured Cells with the Composition of Example 3

Normal mouse embryonic fibroblasts are obtained as in Example 1 above, and are seeded in 10 ml of DMEM, 10% FBS in 10×2 cm, tissue culture treated, polystyrene dishes (Falcon® #353003, Becton Dickinson) at a cell density of 3×10⁵ cells per plate. After 24 hours, the growth media is replaced with fresh media containing of continual growth-inducing composition from Example 3 above, in various concentrations from 50 nM to 300 nM, or an equal volume of the solvent buffer for the compound. Growth media is replaced with fresh media containing the same constituents every 1 to 3 days. Population doubling is assayed as in Example 2. A significant increase in population doubling, along with absence of morphological signs of senescence indicates continual growth has been induced. Reversibility of the induced continual growth is expected on removal of the continual growth-inducing composition from contact with the cultured cells, as evaluated by the significant decrease in the population doublings of the treated cells.

### EXAMPLE 5

### Transformation of Continual Growth-Induced Cultured Cells with Oncogene-Containing Plasmids

The continual growth-induced cultured cells from Example 4 are transfected with pcDNA3 plasmid vectors containing the human oncogenes Ha-ras^{V12}, pE1A, or c-myc. Each oncogenic plasmid is evaluated for the ability to transform the cultured cells by scoring the foci formed after 21 days in culture.

Transfection of Cultured Cells with Plasmids and Scoring of Foci

10⁶ early passage (<4) mouse embryonic fibroblast (MEF) cells are obtained as in Example 1. The growth medium is changed 4-6 hours before transfections are begun. Transfections are performed by standard calcium phosphate or DEAE-Dextran procedures as follows.

Calcium Phosphate Transfection

The calcium phosphate transfection is performed essentially as described in Current Protocols in Molecular Biology (1996), Ausubel et al., eds., John Wiley and Sons, Inc., USA, the disclosure of which is herein incorporated by reference.

Cultured MEFs are collected and seeded at 1.5×10⁴ cells per plate (10×2 cm, tissue culture treated polystyrene dishes; Falcon® #353003, Becton Dickinson) and cultured for 18-24 hours. Approximately 5 μg of circular or linearized plasmid DNA is added to 50 microliters of 2.5M CaCl₂ and the volume is adjusted to 500 microliters with H₂O. Air is bubbled into 500 microliters of 2×HEPES buffer (0.5M HEPES, 5M NaCl, 0.5M Na₂HPO₄) in a 15 ml tube (Falcon Blue Max Jr., #352097, Becton Dickinson) while dropwise adding the 500 microliter DNA/CaCl₂ mixture. After vortexing this solution for ten seconds, the solution is added to the plated cells. After 24 hours, the cells are washed three times with sterile PBS and 10 ml of fresh media is added. Transfected cells are maintained in culture for 21 days, with fresh growth media supplied every three days.

DEAE-Dextran Transfection

DEAE-Dextran transfection is performed essentially as described in Current Protocols in Molecular Biology (1996), Ausubel et al., eds., John Wiley and Sons, Inc., USA supra. Cultured MEFs are collected and seeded at 5×10⁵ cells per plate (10×2 cm, tissue culture treated, polystyrene dishes; Falcon® #353003, Becton Dickinson) and cultured for 18-24 hours. DEAE-Dextran/DNA mix is prepared as follows: 5 ml DMEM (Gibco)/10% NuSerum (Collaborative Research), 200 microliters DNA (1-20 micrograms), 200 microliters DEAE-Dextran/chloroquine (10 mg/ml DEAE/Dextran, 2.5 mM chloroquine in sterile PBS). The plated cells are washed with PBS incubated with 10 ml of the DEAE-Dextran DNA mix for two hours. Cells are washed twice with PBS, and the growth media (DMEM, 10% FBS) is replaced. Transfected cells are maintained in culture for 21 days, with fresh growth media supplied every three days.

Scoring of Foci

At 21 days past transfection, cells are fixed and stained with Giemsa, and foci (>2 mm in diameter) as scored visually.

### EXAMPLE 6

### Preparation of Continual Growth Inducing Composition Comprising cdk4^{R24C} and TERT

cdk4^{R24C}/PTD/6×His fusion protein is obtained as in Example 3 above. TERT fusion protein is produced by cloning the nucleotide sequence of SEQ ID NO: 67 and the NH₂-terminal 11-amino acid PTD from the human immunodeficiency virus TAT protein (SEQ ID NO: 71), in the same reading frame, into a pET-15b expression vector (Novagen) via the multicloning site by standard techniques. This vector allows production of a fusion protein with an N-terminal 6His tag. This pET-15b plasmid construct is used to transform high-expressing bacteria BL21(DE3), and TERT/PTD/6×His fusion protein is isolated from the transfected as in Example 3, above.

The continual growth-inducing composition is made by mixing equimolar amounts of cdk4^{R24C}/PTD/6×His fusion protein and TERT/PTD/6×His fusion protein in physiological saline as a solvent buffer.

### EXAMPLE 7

### Reversible Induction of Continual Growth in Cultured Cells with the Composition of Example 6

Normal mouse embryonic fibroblasts are obtained as in Example 1 above, and are seeded in 10 ml of DMEM, 10% FBS in 10×2 cm, tissue culture treated, polystyrene dishes (Falcon® #353003, Becton Dickinson) at a cell density of 3×10⁵ cells per plate. After 24 hours, the growth media is replaced with fresh media containing of continual growth-inducing composition from Example 6 above in various concentrations from 50 nM to 300 nM, or an equal volume of the solvent buffer for the compound. Growth media is replaced with fresh media containing the same constituents every 1 to 3 days. Population doubling is assayed as in Example 2. A significant increase in population doubling, along with absence of morphological signs of senescence indicates continual growth has been induced. Reversibility of the induced continual growth is expected on removal of the continual growth-inducing composition from contact with the cultured cells, as evaluated by the significant decrease in the population doublings of the treated cells.

### EXAMPLE 8

### Preparation of Continual Growth Inducing Composition Comprising cdk4^{R24C}, Mutant cdk2, Mutant cdk6, and TERT

cdk4^{R24C}/PTD/6×His fusion protein and TERT/PTD/6×His fusion protein are obtained as in Example 6 above. cdk2 and cdk6 fusion proteins having activating mutations, and including a PTD and a 6×His tag are obtained as follows.

Mutant cdk2 fusion protein is made by cloning a nucleic acid sequence encoding cdk2 with in which Arg22 is replaced by Cys (SEQ ID NO: 33) and the NH₂-terminal 11-amino acid PTD from the human immunodeficiency virus TAT protein (SEQ ID NO: 71), in the same reading frame, into a pET-15b expression vector (Novagen) via the multicloning site by standard techniques. This vector allows production of a fusion protein with an N-terminal 6His tag. This pET-15b plasmid construct is used to transform high-expressing bacteria BL21(DE3), and mutant cdk2/PTD/6×His fusion protein is isolated from the transfected as in Example 3, above.

Mutant cdk6 fusion protein is made by cloning a nucleic acid sequence encoding ckd6 in which Arg31 is replaced by Cys (SEQ ID NO: 20) and the NH₂-terminal 11-amino acid PTD from the human immunodeficiency virus TAT protein (SEQ ID NO: 71), in the same reading frame, into a pET-15b expression vector (Novagen) via the multicloning site by standard techniques. This pET-15b plasmid construct is used to transform high-expressing bacteria BL21(DE3), and mutant cdk2/PTD/6×His fusion protein is isolated from the transfected as in Example 3, above.

The continual growth-inducing composition is made by mixing equimolar amounts of cdk4^{R24C}, mutant cdk2, mutant cdk6 and TERT fusion proteins obtained as describe above in physiological saline as a solvent buffer.

### EXAMPLE 9

### Telomeric Repeat Amplification Protocol (TRAP) Assay for Telomerase Activity of Putative TERT Compounds

A putative TERT protein, or fragment, derivative, homolog or analog thereof (hereinafter “TERT compound”) is tested for telomerase activity according to the procedure of Kim et al. (1994), Science 266: 2011-2015, the disclosure of which is herein incorporated by reference.

The TERT compound is mixed with 0.1 microgram of the non-telomeric oligonucleotide TS (5′-AATCCGTCGAGCAGAGTT-3′ (SEQ ID NO: 72)) in a 50 microliter reaction containing 20 mM Tris-HCl (pH 8.3); 1.5 mM EGTA; 50 micromolar A, C, G, T deoxynucleoside triphosphates; 1 microgram of T4g32 protein (Boehringer Mannheim); 0.1 mg/ml bovine serum albumin; 2 units of Taq DNA polymerase (Boehringer Mannheim). 0.2 to 0.4 microliter of alpha-³²P deoxyguanosine triphosphate or alpha-³²P deoxycytidine triphosphate (10 microCurie/microliter) can be added to radiolabel the extension product. The reaction mix is incubated for 10 min. at 23° C. to extend the TS oligonucleotide. CX primer (5′-(CCCTTA)₃CCCTAA-3′ (SEQ ID NO: 73)) is added, and the reaction mix is transferred to a thermal cycler for 27 rounds of PCR amplification at 94° C. for 30 s; 50° C. for 30 s; and 72° C. for 90 s. PCR products are analyzed by electrophoresis in 0.5×Tris-Borate EDTA on 15% polyacrylamide non-denaturing gels. Telomerase activity is shown by the presence of amplified telomeric repeats on the gel. Positive telomerase activity by this assay indicates that the compound being tested in an TERT compound.

All documents referred to herein are incorporated by reference. While the present invention has been described in connection with the preferred embodiments of the various figures, it is to be understood that other similar embodiments can be used or modifications and additions made to the described embodiments for performing the same function of the present invention without deviating therefrom. Therefore, the present invention should not be limited to any single embodiment, but rather should be construed in breadth and scope in accordance with the recitation of the appended claims.

We claim:

1. A composition for inducing a reversible state of continual growth in cultured cells, comprising at least one compound comprising a cdk4, cdk2 or cdk6 protein having an activating mutation, or biologically active fragment, derivative, homolog or analog of the cdk4, cdk2, cdk6 protein, wherein the compound further includes one or more modifications which allow the compound to enter the cells when administered to the cells in culture.

2. The composition of claim 1, wherein the compound comprises a cdk4 protein selected from the group consisting of SEQ ID NO: 1; SEQ ID NO: 3; SEQ ID NO: 5; SEQ ID NO: 7; SEQ ID NO: 9; SEQ ID NO: 11; and SEQ ID NO: 13 having an activating mutation.

3. The composition of claim 1 wherein the compound comprising the cdk4 protein comprises the amino acid sequence of SEQ ID NO: 15.

4. The composition of claim 1, wherein the cdk4 protein activating mutation comprises an exchange of the conserved arginine in the p16^{Ink4a} binding site of the cdk4 protein for any amino acid.

5. The composition of claim 4, wherein the conserved arginine is exchanged for a cysteine.

6. The compound of claim 1, wherein the cdk4 protein comprises the amino acid sequence of SEQ ID NO: 27, and the activating mutation comprises a mutation in the amino acid sequence of SEQ ID NO: 27.

7. The composition of claim 1, wherein the compound comprises a cdk6 protein selected from the group consisting of SEQ ID NO: 17; SEQ ID NO: 21; SEQ ID NO: 23; and SEQ ID NO: 25 having an activating mutation.

8. The composition of claim 1, wherein the cdk6 protein activating mutation comprises an exchange of the conserved arginine in the p16 binding site in the cdk6 protein for any amino acid.

9. The composition of claim 8, wherein the conserved arginine is exchanged for a cysteine.

10. The compound of claim 1, wherein the cdk6 protein comprises the amino acid sequence of SEQ ID NO: 28, and the cdk6 activating mutation comprises a mutation in the amino acid sequence of SEQ ID NO: 28.

11. The composition of claim 1, wherein the compound comprises a cdk4 protein or cdk6 protein comprising the amino acid sequence SEQ ID NO: 29, and the cdk4 or cdk6 activating mutation comprises a mutation in the amino acid sequence of SEQ ID NO: 29.

12. The composition of claim 1, wherein the compound comprises a cdk2 protein selected from the group consisting of SEQ ID NO: 30; SEQ ID NO: 34; SEQ ID NO: 36; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 52; and SEQ ID NO: 54 having an activating mutation.

13. The composition of claim 1 wherein the compound comprises a cdk2 protein which comprises the amino acid sequence of SEQ ID NO: 32.

14. The composition of claim 1, wherein the compound comprises a cdk2 protein comprising the amino acid sequence SEQ ID NO: 55, and the cdk2 activating mutation comprises a mutation in the amino acid sequence of SEQ ID NO: 55.

15. The composition of claim 1 further comprising a compound comprising a TERT protein, or biologically active fragment, derivative, homolog or analog thereof, wherein the compound includes one or more modifications which allow the compound to enter the cells when administered to the cells in culture.

16. The composition of claim 15, wherein the compound is selected from the group consisting of SEQ ID NO: 56; SEQ ID NO: 57; SEQ ID NO: 58; SEQ ID NO: 60; SEQ ID NO: 61; SEQ ID NO: 62; SEQ ID NO: 64; SEQ ID NO: 66; and SEQ ID NO: 68.

17. The composition of claim 1, wherein said one or more modifications which allow the at least one compound to enter a cell comprise a leader sequence which directs entry of the compound into the cell.

18. The composition of claim 17, wherein the leader sequence comprises SEQ ID NO: 70 or SEQ ID NO: 71.

19. A method of inducing a reversible state of continual growth in cultured cells, comprising the steps of:
a) providing a culture of viable cells;
b) contacting the cells with an effective amount of the composition of claim 1; and
c) optionally reversing the state of continual growth by removing the composition from contact with the cells.

20. The method of claim 19, wherein the cells are obtained from amphibians, reptiles, birds, mammals, fish, arthropods or insects.

21. The method of claim 20, wherein the cells are obtained from a mammal.

22. The method of claim 21, wherein the mammal is selected from the group consisting of humans; rodents; rabbits; ovine mammals; bovine mammals; and porcine mammals.

23. The method of claim 19, wherein the cultured cells comprise stem cells.

24. The method of claim 23, wherein the stem cells are selected from the group consisting of hematopoietic stem cells; mesenchymal stem cells; neural stem cells; neural progenitor cells; embryonic stem cells; embryonic primordial germ cells; skeletal muscle satellite cells; and blastocyst inner cell masses.

25. The method of claim 23 wherein the stem cells comprise mammalian stem cells.

26. The method of claim 25 wherein the mammalian stem cells are mouse stem cells.

27. The method of claim 25 wherein the mammalian stem cells are human stem cells.

28. A method of screening an agent for the ability to transform cultured cells, comprising the steps of:
a) providing a culture of viable cells;
b) contacting the cells with an effective amount of the composition of claim 1, so that a state of continuous growth is induced for as long as the cells are in contact with the composition;
c) contacting the cells with an agent; and
d) evaluating the cells for the presence of a transformed phenotype.

29. The method of claim 28, wherein the agent is selected from the group consisting of ionizing radiation, carcinogens, mutagens, teratogens, and nucleic acid sequences.

30. The method of claim 28, wherein the nucleic acid sequences comprise a plasmid containing a gene or gene fragment.

31. A cultured cell in which a reversible state of continual growth has been induced by the composition of claim 1.

32. The cultured cell of claim 31, wherein the cells are obtained from amphibians, reptiles, birds, mammals, fish, arthropods, or insects.

33. The cultured cell of claim 32, wherein the cells are obtained from a mammal.

34. The cultured cell of claim 33, wherein the mammal is selected from the group consisting of humans; rodents; rabbits; ovine mammals; bovine mammals; and porcine mammals.

35. The cultured cell of claim 31, wherein the cultured cells comprise stem cells.

36. The cultured cell of claim 35, wherein the stem cells are selected from the group consisting of hematopoietic stem cells; mesenchymal stem cells; neural stem cells; neural progenitor cells; embryonic stem cells; embryonic primordial germ cells; skeletal muscle satellite cells; and blastocyst inner cell masses.

37. The cultured cell of claim 35 wherein the stem cells comprise mammalian stem cells.

38. The cultured cell of claim 37 wherein the mammalian stem cells are mouse stem cells.

39. The cultured cell of claim 37 wherein the mammalian stem cells are human stem cells.
